SEQUENCE LISTING (56 sequences)

SEQ ID NO: 1 - DNA, 592 bp, Homo sapiens
atgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggct

SEQ ID NO: 2 - DNA, 603 bp, Homo sapiens
atggctctcctgaccaatctactgcccctgtgctgcttggcacttctggcgctgccagcccagagctgcgggccgggccgggggccggttggccggcgccgctatgcgcgcaagcagctcgtgccgctactctacaagcaatttgtgcccggcgtgccagagcggaccctgggcgccagtgggccagcggaggggagggtggcaaggggctccgagcgcttccgggacctcgtgcccaactacaaccccgacatcatcttcaaggatgaggagaacagtggagccgaccgcctgatgaccgagcgttgtaaggagcgggtgaacgctttggccattgccgtgatgaacatgtggcccggagtgcgcctacgagtgactgagggctgggacgaggacggccaccacgctcaggattcactccactacgaaggccgtgctttggacatcactacgtctgaccgcgaccgcaacaagtatgggttgctggcgcgcctcgcagtggaagccggcttcgactgggtctactacgagtcccgcaaccacgtccacgtgtcggtcaaagctgataactcactggcggtccgggcgggcggctgctttccg

SEQ ID NO: 3 - DNA, 600 bp, Homo sapiens
ggagaacacaggcgccgaccgcctcatgacccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccgagcactcggccgcagccaagacaggcggctgcttccctgccggagcccaggtacgcctggagagtggggcgcgtgtggccttgtcagccgtgaggccgggagaccgtgtgctggccatgggggaggatgggagccccaccttcagcgatgtgctcattttcctggaccgcgagcctcacaggctgagagccttccaggtcatcgagactcaggaccccccacgccgcctggcactcacacccgctcacctgctctttacggctgacaatcacacggagccggcagcccgcttccgggccacatt

SEQ ID NO: 4 - DNA, 20 bp, Artificial Sequence
  Description of Artificial Sequence  Synthetic primer
gagcagaccggctgatgact

SEQ ID NO: 5 - DNA, 22 bp, Artificial Sequence
  Description of Artificial Sequence  Synthetic primer
agagatggccaaggcatttaac

SEQ ID NO: 6 - DNA, 15 bp, Artificial Sequence
  Description of Artificial Sequence  Synthetic primer
agaggtgcaaagaca

SEQ ID NO: 7 - DNA, 18 bp, Artificial Sequence
  Description of Artificial Sequence  Synthetic primer
cgcagaccgcctgatgac

SEQ ID NO: 8 - DNA, 20 bp, Artificial Sequence
  Description of Artificial Sequence  Synthetic primer
gcgatggctagagcgttcac

SEQ ID NO: 9 - DNA, 14 bp, Artificial Sequence
  Description of Artificial Sequence  Synthetic primer
agcgttgcaaagag

SEQ ID NO: 10 - DNA, 20 bp, Artificial Sequence
  Description of Artificial Sequence  Synthetic primer
caaaccggctgagagctttc

SEQ ID NO: 11 - DNA, 16 bp, Artificial Sequence
  Description of Artificial Sequence  Synthetic primer
agccgacgcggaggat

SEQ ID NO: 12 - DNA, 16 bp, Artificial Sequence
  Description of Artificial Sequence  Synthetic primer
aggtcatcgagactca

SEQ ID NO: 13 - DNA, 20 bp, Artificial Sequence
  Description of Artificial Sequence  Synthetic primer
cgtcactacctggcctcaca

SEQ ID NO: 14 - DNA, 19 bp, Artificial Sequence
  Description of Artificial Sequence  Synthetic primer
ccccctggctgaagcatat

SEQ ID NO: 15 - DNA, 23 bp, Artificial Sequence
  Description of Artificial Sequence  Synthetic primer
ccagcactacatgctccgggcaa

SEQ ID NO: 16 - DNA, 21 bp, Artificial Sequence
  Description of Artificial Sequence  Synthetic primer
caaaaacgaaagcgcaagaaa

SEQ ID NO: 17 - DNA, 20 bp, Artificial Sequence
  Description of Artificial Sequence  Synthetic primer
cgctctgaacaaggctcaca

SEQ ID NO: 18 - DNA, 25 bp, Artificial Sequence
  Description of Artificial Sequence  Synthetic primer
cccggtttaaatcctggagcgttca

SEQ ID NO: 19 - DNA, 23 bp, Artificial Sequence
  Description of Artificial Sequence  Synthetic primer
cagatacaacagaatgcggttca

SEQ ID NO: 20 - DNA, 23 bp, Artificial Sequence
  Description of Artificial Sequence  Synthetic primer
tgagacaagaggctggttcctat

SEQ ID NO: 21 - DNA, 22 bp, Artificial Sequence
  Description of Artificial Sequence  Synthetic primer
aaccacacggccaccatgctgg

SEQ ID NO: 22 - DNA, 26 bp, Artificial Sequence
  Description of Artificial Sequence  Synthetic primer
ctacaggattcaccttacaggactca

SEQ ID NO: 23 - DNA, 20 bp, Artificial Sequence
  Description of Artificial Sequence  Synthetic primer
cttcctggttggctgatgct

SEQ ID NO: 24 - DNA, 20 bp, Artificial Sequence
  Description of Artificial Sequence  Synthetic primer
tgattttgcccgccgtgcct

SEQ ID NO: 25 - DNA, 22 bp, Artificial Sequence
  Description of Artificial Sequence  Synthetic primer
cctacaaagtcagctcgttcca

SEQ ID NO: 26 - DNA, 22 bp, Artificial Sequence
  Description of Artificial Sequence  Synthetic primer
tccttctgagtcttgggcatgt

SEQ ID NO: 27 - DNA, 18 bp, Artificial Sequence
  Description of Artificial Sequence  Synthetic primer
cgggcccagcgccacact

SEQ ID NO: 28 - DNA, 22 bp, Artificial Sequence
  Description of Artificial Sequence  Synthetic primer
atcagttacggtaagccagtca

SEQ ID NO: 29 - DNA, 19 bp, Artificial Sequence
  Description of Artificial Sequence  Synthetic primer
tggcgacatggctctcaaa

SEQ ID NO: 30 - DNA, 26 bp, Artificial Sequence
  Description of Artificial Sequence  Synthetic primer
ctgagctacagatgcccctgccgatt

SEQ ID NO: 31 - DNA, 22 bp, Artificial Sequence
  Description of Artificial Sequence  Synthetic primer
aaggacaagttgaacgctttgg

SEQ ID NO: 32 - DNA, 18 bp, Artificial Sequence
  Description of Artificial Sequence  Synthetic primer
tcggtcacccgcagtttc

SEQ ID NO: 33 - DNA, 24 bp, Artificial Sequence
  Description of Artificial Sequence  Synthetic primer
ctcctggccactggttcatcaccg

SEQ ID NO: 34 - DNA, 24 bp, Artificial Sequence
  Description of Artificial Sequence  Synthetic primer
caccaccctacctctgtctattcg

SEQ ID NO: 35 - DNA, 22 bp, Artificial Sequence
  Description of Artificial Sequence  Synthetic primer
tcctgtagccccctagtatcca

SEQ ID NO: 36 - DNA, 24 bp, Artificial Sequence
  Description of Artificial Sequence  Synthetic primer
cccagcatcaccgaaaatgttgcc

SEQ ID NO: 37 - DNA, 21 bp, Artificial Sequence
  Description of Artificial Sequence  Synthetic primer
ctctggagcagatttccaagg

SEQ ID NO: 38 - DNA, 20 bp, Artificial Sequence
  Description of Artificial Sequence  Synthetic primer
tgccgcagttcttttgaatg

SEQ ID NO: 39 - DNA, 22 bp, Artificial Sequence
  Description of Artificial Sequence  Synthetic primer
aaggctactggccggaaagcgc

SEQ ID NO: 40 - DNA, 21 bp, Artificial Sequence
  Description of Artificial Sequence  Synthetic primer
catcttcaagccgtcctgtgt

SEQ ID NO: 41 - DNA, 21 bp, Artificial Sequence
  Description of Artificial Sequence  Synthetic primer
cagggcttcatcgttacagca

SEQ ID NO: 42 - DNA, 20 bp, Artificial Sequence
  Description of Artificial Sequence  Synthetic primer
ccgctgatgcgctgtgcagg

SEQ ID NO: 43 - DNA, 19 bp, Artificial Sequence
  Description of Artificial Sequence  Synthetic primer
gggacagcaggcaaacaga

SEQ ID NO: 44 - DNA, 22 bp, Artificial Sequence
  Description of Artificial Sequence  Synthetic primer
tgtcgttatcagcatccttcgt

SEQ ID NO: 45 - DNA, 22 bp, Artificial Sequence
  Description of Artificial Sequence  Synthetic primer
ttgatcttacacggtgccgatt

SEQ ID NO: 46 - DNA, 21 bp, Artificial Sequence
  Description of Artificial Sequence  Synthetic primer
tcagccaaccaggaagtgatt

SEQ ID NO: 47 - DNA, 21 bp, Artificial Sequence
  Description of Artificial Sequence  Synthetic primer
agcatctgggaacacttgcag

SEQ ID NO: 48 - DNA, 29 bp, Artificial Sequence
  Description of Artificial Sequence  Synthetic primer
cacaaaggattcggacaatgacaaatgca

SEQ ID NO: 49 - DNA, 19 bp, Artificial Sequence
  Description of Artificial Sequence  Synthetic primer
cctccaaacgcatgcttca

SEQ ID NO: 50 - DNA, 20 bp, Artificial Sequence
  Description of Artificial Sequence  Synthetic primer
ccttccattgcagcattggt

SEQ ID NO: 51 - DNA, 29 bp, Artificial Sequence
  Description of Artificial Sequence  Synthetic primer
ctgacttccgcttctcacctctgtagcct

SEQ ID NO: 52 - DNA, 16 bp, Artificial Sequence
  Description of Artificial Sequence  Synthetic primer
cgggtcgggagtgggt

SEQ ID NO: 53 - DNA, 21 bp, Artificial Sequence
  Description of Artificial Sequence  Synthetic primer
gaaacggctaccacatccaag

SEQ ID NO: 54 - DNA, 19 bp, Artificial Sequence
  Description of Artificial Sequence  Synthetic primer
tttgcgcgcctgctgcctt

SEQ ID NO: 55 - DNA, 675 bp, Homo sapiens
  CDS (1)..(675)
ggcgagatgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggctgcttcccgggctcggccacggtgcacctggagcagggcggcaccaagctggtgaaggacctgagccccggggaccgc

SEQ ID NO: 56 - protein, 225 aa, Homo sapiens
GEMLLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDR

(19) United States
(12) Patent Application Publication
(10) Pub. No.: US 2006/0105950 A1
(43) Pub. Date: May 18, 2006

(54) Morphogen compositions and use thereof to treat wounds

(75) Inventor: Douglas W. Losordo, Winchester, MA (US)

Correspondence Address: EDWARDS & ANGELL, LLP, P.O. BOX 55874, BOSTON, MA, 02205, US

(73) Assignee: Caritas St. Elizabeth's Medical Center of Boston, Inc.

(21) Appl. No.: 11/254,524

(22) Filed: Oct. 20, 2005

Related U.S. Application Data
(60) Provisional application No. 60/621,772, filed on Oct. 25, 2004.

(51) Int. Cl. A61K 38/17; A61K 38/18; A61K 48/00

(52) U.S. Cl. 514/12; 514/44

(57) ABSTRACT

Disclosed are compositions and methods for promoting or accelerating wound healing and preventing, treating, or reducing symptoms associated with wounds or wounding disorders such as diabetic ulcers and burns. In one embodiment, the method includes administering a therapeutically effective amount of a nucleic acid encoding at least one morphogen, or an effective fragment thereof. Preferred morphogens include the human Sonic Hedghog (Shh), human Desert Hedgehog (Dhh), and human Indian Hedgehog (Ihh) proteins. The methods can be used alone or in combination with other methods involving administration of an angiogenic protein, a hematopoietic protein, or cells such as endothelial cells or endothelial precursor cells.

### CROSS-REFERENCE TO RELATED APPLICATION

The present application claims priority from U.S. Provisional Patent Application No. 60/621,772 entitled Morphogen Compositions and Use Thereof To Treat Wounds, filed on Oct. 25, 2004, the disclosure of which is herein incorporated by reference in its entirety.

### STATEMENT AS TO FEDERALLY SUPPORTED RESEARCH

The present invention was made with United States government support under National Institutes of Health (NIH) grant number HL 53354. Accordingly, the United States government may have certain rights to the invention.

### FIELD OF THE INVENTION

The invention generally relates to compositions and methods for preventing, treating or reducing the severity of a wound or wounding disorder. In one aspect, the method includes administering to a mammal a therapeutically effective amount of a nucleic acid encoding at least one morphogen or an effective fragment thereof, or a nucleic acid encoding the same, to prevent or treat the disorder.

### BACKGROUND OF THE INVENTION

The skin is a multi-layered organ made of up several tissue layers including the outermost epidermis which serves a protective function, and several supporting connective tissue layers (dermis and hypodermis) that inter alia provide essential nutrition to the epidermal cells by means of an extensive vascular network. In many chronic medical conditions and other situations requiring medical attention, the skin is subjected to various forms of wounds, including burns, bedsores, traumatic and surgical wounds, and ulcers. Ulcers of various types affecting the skin are particularly common in conditions such as diabetes, in which the ability of the skin to heal is compromised. In diabetes and many other medical conditions, poor oxygen delivery particularly to the limbs, or immobile body parts (for example in bedridden patients or those in wheelchairs), results in slow healing, infections, scar development, and in the worst cases, tissue death requiring amputation. Non-healing skin ulcers are one of the most serious consequences of diabetes, resulting in more hospitalizations than any other diabetic complication (Singer et al., 1999). Of the various factors that may contribute to the non-healing of wounds in this disorder, impaired angiogenesis (formation of new blood vessels) is thought to play a central role.

A particularly serious form of skin wounding condition is burn. According to the American Burn Association, each year in the United States, 1.1 million burn injuries require medical attention. Approximately 50,000 of these require hospitalization, and roughly half of those burn patients are admitted to a specialized burn unit. Each year, approximately 4,500 of these people die. Additionally, up to 10,000 people in the United States die every year of burn-related infections, with pneumonia being the most common infectious complication among hospitalized burn patients.

Burn is defined as tissue damage caused by a variety of agents, such as heat, chemicals, electricity, sunlight, or nuclear radiation. Most common are burns caused by scalds, building fires, and flammable liquids and gases. First-degree burns affect only the epidermis of the skin, whereas more serious second-degree burns damage both the epidermis and the dermis, and third-degree burns involve damage or complete destruction of the skin and damage to underlying tissues. The swelling and blistering characteristic of burns are caused by the loss of fluid from damaged blood vessels. In severe cases, such fluid loss can cause shock, requiring immediate transfusion of the patient with blood or a physiological salt solution to restore adequate fluid levels to maintain blood pressure.

Like ulcers, burns often lead to infection, due to damage to the skin's protective barrier. In some cases, topical antibiotics (creams or ointments applied to the skin) can prevent or treat such infection. The three topical antibiotics that are most widely used are silver sulfadiazene cream, mafenide acetate cream, and silver nitrate.

Therapeutic advances have contributed to improvements in treatments for wounding disorders such as non-healing diabetic wounds, ulcers and burns. For example, there have been advances in resuscitation, wound cleaning and follow-up care, nutritional support, and infection control. Grafting with natural or artificial materials can also speed the healing process. Current treatments for burns and other slow-healing wounds also include exposure to hyperbaric oxygen or superoxygenated fluids.

Despite these advances, it is recognized that improving methods of wound healing and tissue repair remains an ongoing need to enhance the quality of life for trauma, burn and diabetic patients. In the search for new therapies, it is increasingly appreciated that a potential exists to utilize signaling pathways active during embryonic development to effect therapeutic repair mechanisms in adult tissues. An exemplary pathway of this type is activated by genes of the Hedgehog (Hh) gene family, originally reported in Drosophila as a critical regulator of cell-fate determination during embryogenesis (Nusslein-Volhard C, 1980). Hh genes act as morphogens in a wide variety of tissues during embryonic development (Wang et al 1995; Roelink et al. 1994; Goodrich and Scott, 1998) primarily via actions upon mesoderm in epithelial/mesenchymal interactions that are crucial to limb, lung, gut, hair follicle and bone formation (Johnson 1997; Pepicelli et al. 1998; Ramalho-Santos et al. 2000; St-Jacques et al. 1998, 1999).

Three members of the mammalian Hh family have been reported. They are Sonic hedgehog (Shh), Desert hedgehog (Dhh) and Indian hedgehog (Ihh). Among these three highly conserved mammalian Hh genes, Sonic hedgehog (Shh) is the most widely expressed during development and the best studied (Zardoya et al. 1996; Bitgood et al. 1995). Hh signaling occurs through the interaction of Hh ligand with its receptor, Patched-1 (Ptc-1) (Ingham, 1998). Hh binds the Ptc-1 receptor, with subsequent activation of Smoothened (Smo). Activation of Smo initiates signaling events that lead to the regulation of transcription factors belonging to the Gli family, which modify the expression of downstream target genes of the Hh pathway (Kogerman et al. 1999; Sisson et al. 1997; Pola et al., 2001).

It would be desirable to use at least one of the Sonic hedgehog (Shh), Desert hedgehog (Dhh) and Indian hedgehog (Ihh) proteins (or effective fragments thereof) to prevent or treat wounds or wounding disorders. It would be particularly useful to administer a nucleic acid encoding the human Shh protein to prevent or treat wounds and related conditions in a human patient.

### SUMMARY OF THE INVENTION

The invention generally relates to a method for preventing or treating a wound or wounding disorder in a mammal, or related medical indication. In one aspect, practice of the invention involves administering to the mammal a therapeutically effective amount of at least one morphogenic protein or an effective fragment thereof, or a nucleic acid encoding such a morphogenic protein or fragment. The mammal can be one that has, is suspected of having, or is at risk of developing a condition or disorder involving wounding.

We have discovered that wounds and wounding disorders can be treated by administering a therapeutically effective amount of at least one nucleic acid that encodes at least one morphogen, or an effective fragment thereof. In preferred embodiments, the morphogen is human sonic hedgehog (Shh) protein, desert hedgehog (Dhh) protein, or Indian hedgehog (Ihh) protein.

The invention thus provides a new strategy for preventing, treating, or reducing the severity of particular disorders, especially wounding disorders and related ailments. Disorders and conditions that may particularly benefit from the methods and compositions of the invention include wounds due to trauma or surgery and various types of ulcers including diabetic ulcers, pressure (decubitus) ulcers and ulcers due to vascular insufficiency. Without wishing to be bound to any particular theory, it has been found that administration of a morphogen according to the invention facilitates a downstream cascade of one or more desirable cytokines and angiogenic factors which can promote wound healing, increase angiogenesis and help reduce the severity or healing times in wounding disorders such as diabetes.

The invention is flexible and can be used alone or in combination with other therapies as needed. As described below, such therapies include, but are not limited to, direct administration to the mammal of a solution that includes the nucleic acid, either alone or together with administration of at least one of a morphogenic, angiogenic, or hematopoietic protein, or an effective fragment thereof. The invention further provides for administration of at least one of endothelial cells (ECs) and endothelial precursor cells (EPCs), which is believed to assist practice of the invention in certain settings.

Accordingly, and in one aspect, the invention provides a method for preventing, treating or reducing the severity of a wound or wounding disorder in a mammal. In one embodiment, the method comprises administering a therapeutically effective amount of at least one morphogenic protein or effective fragment thereof, or a nucleic acid encoding the morphogen or fragment. Typical methods include selecting a mammal having a wound or wounding disorder and administering the nucleic acid directly to or near a wound in need of treatment. A preferred mammal is a rodent or primate, including a human patient.

As discussed, the invention features a method for preventing or treating a wound or wounding disorder or accelerating wound healing that includes administering an effective morphogenic fragment. Particular fragments are discussed herein and include the N-terminal portion of the human hedgehog (Shh) protein, human desert hedgehog (Dhh) protein or human Indian hedgehog (Ihh) protein. As shown below, the method can be used to promote wound healing, including accelerating wound closure, increasing thickness of the healing skin and increasing vascularity of the wounded area. These and other features of the invention provide several benefits including providing a new therapeutic approach to treating wounds and wounding disorders including acute injuries such as those caused by trauma or surgical procedures, and chronic forms of poorly healing wounds such as ulcers and bedsores associated with a wide variety of disorders and conditions including diabetes, conditions involving vascular insufficiency, and immobility in bedridden patients.

Further provided by the present invention is a pharmaceutical product for preventing or treating a wound or wounding disorder or accelerating wound healing in a mammal. In one embodiment, the product includes at least one morphogenic protein or effective fragment thereof, or a nucleic acid encoding the morphogenic protein, formulated to be physiologically acceptable to a mammal and suitable for topical administration. The nucleic acid in the pharmaceutical product can, in one embodiment, be in the form of a plasmid or viral vector suitable for gene therapy. For some invention embodiments, the morphogen-encoding nucleotide sequence can encode a fragment of the morphogenic protein that is secreted from a cell. In preferred embodiments of the product, the morphogen or effective fragment thereof is selected from human sonic hedgehog, human desert hedgehog and human Indian hedgehog protein. Typical products of the invention are sterile and can further include at least one angiogenic or hematopoietic protein, or a nucleic acid encoding these proteins, and optionally at least one of endothelial cells (EC) or endothelial precursor cells (EPC).

Also provided by the present invention is a kit for the administration of at least one morphogenic protein to a mammal. In one embodiment, the kit includes at least one morphogenic protein or effective fragment thereof, or a nucleic acid encoding same, formulated for topical application. Kits according to the invention include a pharmacologically acceptable carrier and directions for using the kit.

In another aspect, the invention relates to a method for inducing new blood vessel formation in the skin of a mammal in need of such treatment. In one embodiment, the method includes administering a therapeutically effective amount of at least one morphogen, or an effective fragment thereof, or a nucleic acid encoding the morphogen. Typically preferred invention methods include selecting a patient having the disorder and administering the morphogenic protein or nucleic acid directly to or near a wound in need of treatment. In some embodiments, the method further comprises expressing the morphogenic protein or fragment in or near a wound in the mammal to prevent or treat the wounding disorder.

The invention further provides a method for increasing incorporation of endothelial precursor cells (EPCs) into blood vessels in the skin of a mammal. The method includes contacting the skin of the mammal with an effective amount of at least one morphogenic protein or effective fragment thereof, or a nucleic acid encoding same.

Further provided by the invention is a method for increasing production of at least one cytokine by skin cells of a mammal. In one embodiment, the method involves contacting the cells with an effective amount of at least one morphogenic protein or effective fragment thereof, or a nucleic acid encoding same. In one variation, the method includes administering a therapeutically effective amount of a nucleic acid encoding an N-terminal portion of at least one of human sonic hedgehog, human desert hedgehog or human Indian hedgehog protein. Preferred cytokines that can be upregulated by the inventive method include but are not limited to Glc-1, Ptch1, vascular endothelial growth factor (VEGF), angiopoietin-1, and SDF-1α. Practice of the method can be in vitro (for example, in a culture of a skin graft or tissue explant, or in a skin cell, such as a dermal fibroblast), or in vivo.

Another aspect of the invention is a method for increasing proliferation by skin cells of a mammal, comprising contacting the cells with an effective amount of at least one morphogenic protein or effective fragment thereof, or a nucleic acid encoding same. Various embodiments of the method can be practiced in vitro and in vivo.

These and other objects, advantages, and features of the invention will become apparent to those persons skilled in the art upon reading the details of the invention as more fully described below.

### BRIEF DESCRIPTION OF THE DRAWINGS

FIG. 1 is a photomicrograph showing X-gal positive cells expressing Ptc1 receptor in hair follicles, and surrounding blood vessels in normal skin.

FIGS. 2A-C are three photographs showing Ptc1 receptor expression (X-gal staining), in normal control skin (2A) and in a healing skin wound at 1 day (2B) and 3 days (2C) after wounding.

FIGS. 3A and 3B are two photographs showing abundant Ptc1 expressing cells (X-gal positive cells) near the edge of a wound 3 days after wounding of the skin (3A) but not in control skin (3B).

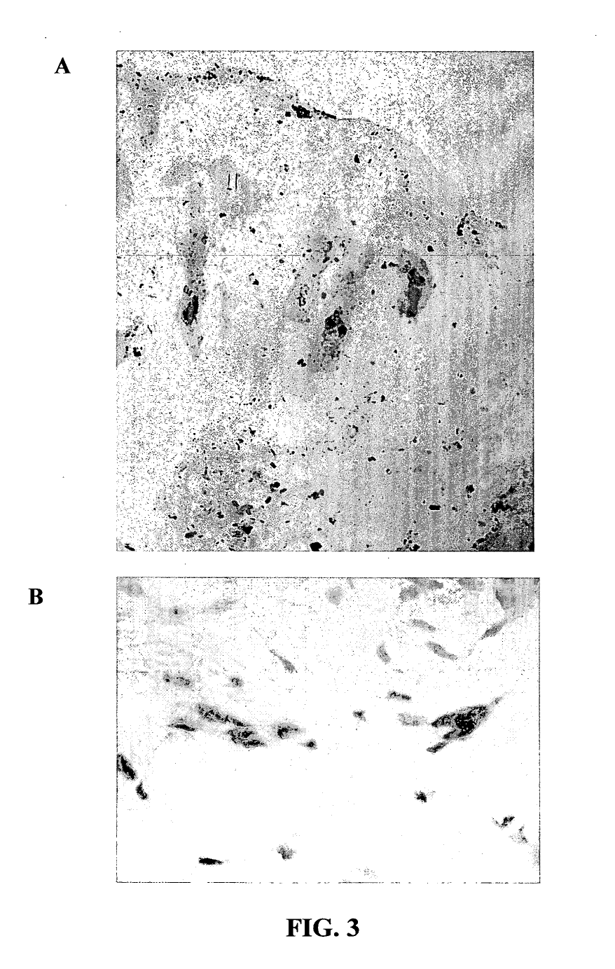

FIGS. 4A-C are two photographs of immunoblots (4A), a graph showing quantitative analysis of hShh protein expression (4B), and a graph showing RT-PCR analysis of hShh mRNA expression (4C), in wounds of wild type and diabetic mice, after topical application of plasmid containing hShh gene (phShh) or control plasmid.

FIGS. 5A-C are four photographs showing the appearance of skin wounds at day 0 (5A) and day 14 (5B) after topical application of phShh or control plasmid, and a graph (5C) showing a significant increase in percentage of wound closure at each time point analyzed after topical application of phShh.

FIGS. 6A-G are six photographs showing the macroscopic (6A and 6B) and microscopic (6C-F) appearance of skin wounds observed 10 days after treatment with control plasmid (6A, 6C, 6E) or phShh (6B, 6D, 6F), and a graph (6G) showing increased skin thickness in a healing wound treated with phShh gene therapy.

FIGS. 7A-C are two photomicrographs (7A and 7B) showing fluorescently labeled blood vessels in skin wounds in diabetic mice 14 days after treatment with control plasmid (7A) or phShh (7B), and a graph (7C) showing a significant increase in vascular density at 14 days wounds treated with phShh.

FIGS. 8A-G are six fluorescence micrographs showing GFP⁺ cells (8A, 8B), cells positive for the endothelial marker BS1 lectin (8C, 8D) and merged images (8E, 8F) showing cells positive for both GFP and BS1, and a graph (8G) showing quantitation of doubly positive cells in new blood vessels formed in healing skin wounds of mice treated topically with phShh plasmid or control plasmid.

FIGS. 9A-E are five graphs showing the effect of Shh protein on expression levels of multiple cytokines in dermal fibroblasts in vitro.

FIG. 10 is a graph showing that Shh protein promotes proliferation of cultured dermal fibroblasts.

FIGS. 11A-D are four graphs showing effects of Shh protein on endothelial precursor cells (EPCs), including upregulating Ptc-1 expression (11A), and promoting EPC migration (11B), proliferation (11C) and adhesion to extracellular matrix components (11D).

FIG. 12 is a diagram showing the nucleic acid sequence of the amino terminal domain of a human sonic hedgehog cDNA and the encoded polypeptide sequence.

### DETAILED DESCRIPTION OF THE INVENTION

As discussed, the invention can be used to prevent, treat, or reduce the severity of a wound or wounding disorder or a related ailment or accelerate wound healing in a mammal such as a human patient. Typical invention methods include administering a therapeutically effective amount of at least one nucleic acid to a patient in need of such treatment, which nucleic acid encodes at least one morphogen, or an effective fragment of that morphogen. Sometimes but not exclusively, the method will involve administering a morphogenic protein or an effective fragment thereof, or a nucleic acid encoding said protein directly to or near a wound in need of treatment in the patient.

As used herein, the term “wound” refers to damage to the integrity of all or a portion of a tissue or cellular layer forming either an external or internal boundary layer in the body of an animal. Examples of tissues forming an external boundary layer include the skin, which as discussed is a multi-layered structure comprised of a superficial layer (the epidermis) and underlying deeper layers (dermis and hypodermis), and the cornea of the eye. Tissues forming internal boundary layers include the epithelial layers and associated mucosal surfaces lining the mouth, nose, ear, alimentary canal, gastrointestinal tract, respiratory tract, reproductive tract and urinary tract, as well as endothelia and underlying connective tissues that line blood vessels including veins, venules, arteries, and arterioles. “Wounding disorders” or “wounding conditions,” as used herein, refer to any medical condition or circumstance that causes damage to the integrity of all or a portion of a tissue or cellular layer forming either an external or internal boundary layer in the body. As described above, well known wounding conditions or circumstances that result in damage to the integrity of a body surface include burns, abrasions, traumatic injuries, surgical wounds, and the like. Certain “wounding disorders” such as vascular insufficiency and diabetes can lead to the breakdown of surface tissues, resulting in formation of poorly healing lesions such as ulcers on the skin, and on internal surfaces such as the lining of the gastrointestinal tract and in blood vessels.

In broad terms, morphogens are recognized amino acid sequences whose concentration is read by cells as positional information relative to a pre-determined landmark or beacon in certain cells. Such sequences help control pattern formation in a large field of adjacent tissue. See generally Alberts, B. et al. (1989) in the Molecular Biology of the Cell, 2^{nd} Ed. Garland Publishing, New York, N.Y. (discussing morphogen function). As used herein, “morphogen” or related phrases such as “morphogenic protein” means one of the following mammalian proteins: Sonic hedgehog (Shh), Desert hedgehog (Dhh) and Indian hedgehog (Ihh), or an effective fragment thereof. Preferred morphogens are endogenous to a primate and especially a human. A more preferred morphogen is human Shh.

There are reports that most morphogens and especially Shh are protein signaling molecules important for vertebrate patterning during development. In particular, Shh has been shown to be involved in the morphogenesis of many vertebrate organ systems.

The sequence of many mammalian morphogens has been disclosed. For example, the human Shh sequence (nucleic acid and protein) has been reported, for example, by Margio, V. (1995) as a submission to the National Center For Biotechnology Information (NCBI) Genetic Sequence Data Bank (Genbank) at the National (U.S) Library of Medicine, 38A, 8N05, Rockville Pike, Bethesda, Md. 20894. See, for example, Accession number L38518 (version L38518.1; GI: 663156), which reports a 1576 base pair human Shh nucleic acid sequence and the corresponding encoded protein. It will be appreciated that any nucleic acid or protein sequence information not specifically disclosed herein can be readily accessed at Genbank, EMBL and/or SWISS-PROT. Using this information, a DNA or RNA segment encoding the desired protein may be chemically synthesized or, alternatively, such a DNA or RNA segment may be obtained using routine procedures in the art, for example, PCR amplification. See also Pola, R. et al. (2003) Circulation 108: 479-485, in which an especially preferred Shh vector construct is disclosed.

By the phrase “effective fragment” or “therapeutically effective fragment” is meant a nucleic acid or amino acid sequence that has at least 80% of the biological function of the corresponding full-length sequence, preferably at least about 90% and more preferably at least about 95% of that function. By an “effective fragment” of a morphogen or related phrase is meant a portion of a morphogenic protein, or nucleic acid encoding the same, that has at least 80% of the activity of the corresponding full-length protein or nucleic acid as determined by what is referred to herein as a “standard wound healing assay.” A preferred version of that assay includes performing at least one of and preferably all of the following steps:
(a) preparing a wound, preferably a full thickness excisional wound, to the skin of a mammal such as a rodent (mouse or rat);
(b) applying to the wound, preferably topically, a formulation such as a solution that includes therein a nucleic acid (about 1 μg/ml to about 5 mg/ml) encoding the desired portion of the morphogen, or optionally the desired morphogenic protein or effective fragment thereof;
(c) allowing the rodent to recover from the wound, i.e., to undergo a process of wound healing for at least about one day or more, and more preferably about a few weeks up to about one to two months; and
(d) evaluating healing of the wound in the mammal, for example by performing: measurement of the wound area, histological assessment of tissue repair, determination of vascular density, detection of expression of cytokines, recruitment of endothelial precursor cells (EPCs), or related procedures, compared to wound healing in control animals.

Preferred embodiments of the assay are discussed below in the Examples section.

Also contemplated for use with the present invention are morphogen “derivatives” which include a morphogen analogue having substantial identity to the corresponding full-length morphogen. Preferred derivatives are at least about 90% identical to the full-length morphogen as determined, for example, by inspection or with the aid of a suitable computer program such as BLAST, FASTA or related programs, preferably at least about 95% identical. Suitable analogues include protein sequences having one or more conservative amino acid substitutions with respect to the corresponding full-length morphogen (for example, allelic variants). By “conservative” amino acid substitution is meant replacement of one amino acid residue for another having similar chemical properties (for example, replacing tyrosine with phenylalanine).

In one embodiment, the method includes administering a therapeutically effective amount of a nucleic acid encoding an N-terminal portion of at least one of human hedgehog (Shh) protein, human desert hedgehog (Dhh) protein and human Indian Hedgehog (Ihh) protein.

It has been reported that Human Shh is synthesized as a 45 kDa precursor protein that is cleaved autocatalytically to yield two “effective fragments” i.e., (1) a 20 kDa N-terminal fragment that is responsible for all known hedgehog signaling activity, and (2) a 25 kDa C-terminal fragment that contains the autoprocessing activity. The N-terminal fragment is reported to consist of amino acid residues 24-197 of the full-length precursor sequence. The N-terminal fragment is thought to remain membrane-associated through the addition of a cholesterol at its C-terminus. The addition of the cholesterol is catalyzed by the C-terminal domain during the processing step. See for example, U.S. Pat. Nos. 6,132,728; 6,281,332, 6,444,793, and 6,288,048 as well as WO 95/18856, and WO 96/17924.

The complete amino acid sequences of the human Dhh and human Ihh proteins have been reported by Genbank as Accession Numbers BC033507 and BC034757, respectively. Also disclosed are the corresponding cDNA sequences. Significant relationship between the N-terminal fragments of the human Shh, human Dhh, and human Ihh proteins has been reported. See U.S. Pat. No. 6,444,793, for instance.

By the term “N-terminal portion” of the human Shh protein is meant between from about 400 bp to about 700 bp amino terminal domain coding sequence of the human Shh gene, preferably about the 600 bp amino terminal domain coding sequence. See Table I below, for instance. By the phrase “N-terminal portion” of the human Dhh and human Ihh proteins is meant less than about an 800 bp amino terminal domain coding sequence, preferably less than about 700 bp, more preferably between from about 300 bp to about 600 bp of the amino terminal domain coding sequence for the human Dhh and human Ihh proteins. Fragment lengths are generally measured from the N-terminus of the mature protein (before autocatalysis). Various other effective N-terminal fragments that encompass the N-terminal moiety are considered within the presently claimed invention. Publications disclosing these sequences, as well as certain of their chemical and physical properties, include WO 95/23223; WO 95/18856; WO 96/17924; WO/99/20298 and U.S. Pat. No. 6,444,793; and U.S. Pat. No. 6,639,051. Preferred of such encoded N-terminal portions of the human Shh, Dhh, and Ihh proteins are those that exhibit at least 80% of the activity of the corresponding full-length protein as determined by the standard wound healing assay described above.

Further effective portions of the human Shh, Dhh, and Ihh proteins are contemplated and include those are at least 80% identical to the N-terminal portion of the proteins specified above, preferably at least about 90% identical or more such as 95% identical. Specifically included are allelic variants of such proteins as well as conservative amino substitutions of such proteins (for example, alanine for serine, glycine for alanine, etc.).

See the following references for disclosure relating to still further morphogens and effective fragments thereof suitable for use with the invention. Lee et al. (1994) Science 266:1528-1537; Porter et al. (1995) Nature 374:363-366); Bumcrot, D. A., et al. (1995) Mol. Cell. Biol. 15:2294-2303; Ekker, S. C. et al. (1995) Curr. Biol. 5:944-955; and Lai, C. J. et al. (1995) Development 121:2349-2360).

Table I below, shows the amino acid sequence of an especially preferred N-terminal amino portion of the human Shh gene sequence (A). Illustrative N-terminal portions of the human Dhh (B) and human Ihh gene (C) sequences are also shown.

Sequence for (B) and (C) was taken from Genbank Accession Nos. BC033507 and BC034757, respectively. In the case of the human Ihh protein sequence, the first amino amino acid in the mature protein sequence is reported by Genbank to be glutamic acid.

TABLE I
Illustrative N-terminal portions
of human Shh, Dhh, and Ihh genes

| | |
|---|---|
| A. | atgctgctgc tggcgagatg tctgctgcta gtcctcgtct cctcgctgct ggtatgctcg ggactggcgt |
| | gcggaccggg cagggggttc gggaagagga ggcaccccaa aaagctgacc cctttagcct acaagcagtt |
| | tatccccaat gtggccgaga agaccctagg cgccagcgga aggtatgaag ggaagatctc cagaaactcc |
| | gagcgattta aggaactcac ccccaattac |
| | aaccccgaca tcatatttaa ggatgaagaa aacaccggag cggacaggct gatgactcag aggtgtaagg |
| | acaagttgaa cgctttggcc atctcggtga tgaaccagtg gccaggagtg aaactgcggg tgaccgaggg |
| | ctgggacgaa gatggccacc actcagagga gtctctgcac tacgagggcc gcgcagtgga catcaccacg |
| | tctgaccgcg accgcagcaa gtacggcatg ctggcccgcc tggcggtgga ggccggcttc gactgggtgt |
| | actacgagtc caaggcacat atccactgct cggtgaaagc agagaactcg gtggcggcca aatcgggagg |
| | ct (SEQ ID NO.1) |
| B. | atg gctctcctga ccaatctact gcccctgtgc tgcttggcac ttctggcgct gccagcccag |
| | agctgcgggc cgggccgggg gccggttggc cggcgccgct atgcgcgcaa gcagctcgtg ccgctactct |
| | acaagcaatt tgtgcccggc gtgccagagc ggaccctggg cgccagtggg ccagcggagg ggagggtggc |
| | aaggggctcc gagcgcttcc gggacctcgt gcccaactac aaccccgaca tcatcttcaa ggatgaggag |
| | aacagtggag ccgaccgcct gatgaccgag cgttgtaagg agcgggtgaa cgctttggcc attgccgtga |
| | tgaacatgtg gcccggagtg cgcctacgag tgactgaggg ctgggacgag gacggccacc acgctcagga |
| | ttcactccac tacgaaggcc gtgctttgga catcactacg tctgaccgcg accgcaacaa gtatgggttg |
| | ctggcgcgcc tcgcagtgga agccggcttc gactgggtct actacgagtc ccgcaaccac gtccacgtgt |
| | cggtcaaagc tgataactca ctggcggtcc gggcgggcgg ctgctttccg (SEQ ID NO. 2) |
| C. | ggagaacaca ggcgccgacc gcctcatgac ccagcgctgc aaggaccgcc tgaactcgct ggctatctcg |
| | gtgatgaacc agtggcccgg tgtgaagctg cgggtgaccg agggctggga cgaggacggc caccactcag |
| | aggagtccct gcattatgag ggccgcgcgg tggacatcac cacatcagac cgcgaccgca ataagtatgg |
| | actgctggcg cgcttggcag tggaggccgg ctttgactgg gtgtattacg agtcaaaggc ccacgtgcat |
| | tgctccgtca agtccgagca ctcggccgca gccaagacag gcggctgctt ccctgccgga gcccaggtac |
| | gcctggagag tggggcgcgt gtggccttgt cagccgtgag gccgggagac cgtgtgctgg ccatggggga |
| | ggatgggagc cccaccttca gcgatgtgct cattttcctg gaccgcgagc ctcacaggctgagagccttc |
| | caggtcatcg agactcagga ccccccacgc cgcctggcac tcacacccgc tcacctgctc tttacggctg |
| | acaatcacac ggagccggca gcccgcttcc gggccacatt (SEQ ID NO 3) |

In embodiments in which prevention or treatment of a wounding disorder is desired, the invention can further include selecting a patient having the disorder and administering the morphogenic protein or nucleic acid directly to or near a wound in need of treatment.

In one approach, and to simplify the manipulation and handling of the nucleic acid encoding the morphogen or effective fragment, the nucleic acid is preferably inserted into a cassette where it is operably linked to a promoter. In the context of gene therapy, the term “effective amount,” as used herein, and especially a “therapeutically effective amount” means a sufficient amount of nucleic acid delivered to the desired cells or tissue in the skin to produce an adequate level of expression of the morphogenic protein or effective fragment thereof, i.e., levels capable of promoting wound healing in general and in particular at least one of blood vessel growth, upregulation of angiogenic factors and cytokines in the wounded tissue, and recruitment of endothelial precursor cells.

Accordingly, an important aspect is the level of morphogen expressed. One can use multiple transcripts for this purpose, or one can have the nucleic acid coding sequence under the control of a promoter that will result in high levels of expression. For instance, administration of between about 1 pg/g organ weight to 1 mg/organ weight will suffice for many invention embodiments.

The promoter must be capable of driving expression of the morphogen in the desired target host cell. The selection of appropriate promoters can readily be accomplished. An example of a promoter suitable to achieve high levels of expression is the 763-base-pair cytomegalovirus (CMV) promoter. The Rous sarcoma virus (RSV) (Davis, et al., Hum Gene Ther 4:151 (1993)) and MMT promoters may also be used. Certain proteins can expressed using their native promoter. Other elements that can enhance expression can also be included such as an enhancer or a system that results in high levels of expression such as a tat gene and tar element.

The cassette comprising at least one nucleic acid encoding a morphogen can be inserted into a vector, for example, a plasmid vector such as pUC 118, pBR322, or other known plasmid vectors, that include, for example, an E. coli origin of replication. See, for example, Sambrook, et al., Molecular Cloning: A Laboratory Manual, Cold Spring Harbor Laboratory press, (1989), and Examples below. The plasmid vector may also include a selectable marker such as the beta-lactamase gene for ampicillin resistance, or a gene conferring neomycin/kanamycin resistance, provided that the marker polypeptide does not adversely affect the metabolism of the organism being treated. The cassette can also be bound to a nucleic acid binding moiety in a synthetic delivery system, such as the system disclosed in WO 95/122618.

If desired, the DNA may also be used with a microdelivery vehicle such as cationic liposomes and adenoviral vectors. For a review of the procedures for liposome preparation, targeting and delivery of contents, see Mannino and Gould-Fogerite, Bio Techniques, 6:682 (1988). See also, Feigner and Holm, Bethesda Res. Lab. Focus, 11 (2):21 (1989) and Maurer, R. A., Bethesda Res. Lab. Focus, 11 (2):25 (1989).

In an alternative embodiment, replication-defective recombinant adenoviral vectors, can be produced in accordance with known techniques. See, for example, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

If desired, the methods disclosed herein can be used alone or in combination with other recognized therapies that promote blood vessel growth. See, for example, EP1061800 and WO99/45775 (disclosing, for example, compositions and method for modulating vascularization). In one embodiment, such methods include administering to the wound of a human patient in need of such treatment at least one nucleic acid encoding at least one of an angiogenic or hemaotpoietic protein, or effective fragment thereof. Methods for testing and identifying a variety of suitable angiogenic and hematopoietic protein fragments have been reported in EP1061800 and WO99/45775, for instance.

As used herein, “angiogenic protein” refers to any protein or fragment thereof that promotes formation of blood vessels. Preferred angiogenic proteins include but are not limited to acidic fibroblast growth factor (aFGF), basic fibroblast growth factor (bFGF), vascular endothelial growth factor (VEGF-1), epidermal growth factor (EGF), transforming growth factor α and β (TGF-α and TFG-β, platelet-derived endothelial growth factor (PD-ECGF), platelet-derived growth factor (PDGF), tumor necrosis factor α (TNF-α), hepatocyte growth factor (HGF), insulin like growth factor (IGF), erythropoietin, colony stimulating factor (CSF), macrophage-CSF (M-CSF), angiopoetin-1 (Ang1) or nitric oxidesynthase (NOS), or an effective fragment thereof.

“Hematopoietic proteins” are defined as any protein or effective fragment thereof that stimulates development, differentiation and/or proliferation of blood cell precursors. Preferred hematopoietic proteins include granulocyte-macrophage colony-stimulating factor (GM-CSF), VEGF, Steel factor (SLF, also known as Stem cell factor (SCF)), stromal cell-derived factor (SDF-1), granulocyte-colony stimulating factor (G-CSF), HGF, angiopoietin-1, angiopoietin-2, M-CSF, b-FGF, and FLT-3 ligand.

Once administered to or near the site of a wound, the nucleic acid (usually DNA) is expressed by the cells in the healing wound (for example, cells of the epidermis, dermis and hypodermis, including but not limited to epithelial cells, melanocytes, macrophages, fibroblasts, neurons, adipocytes, and endothelial cells) for a period of time sufficient to promote wound healing. Methods for testing expression of the morphogenic mRNAs and proteins are known in the art; specific procedures using immunoblot and RT-PCR analysis are further described in Examples below.

Certain vectors in accordance with the invention may not normally be incorporated into the genome of the cells. In these embodiments, expression of the morphogen or effective fragment thereof of interest takes place for only a limited time. In such embodiments, the morphogen or fragment is only expressed in therapeutic levels for about two days to several weeks, for instance about 1-2 weeks up to about one to two months. Reapplication of the DNA can be utilized to provide additional periods of expression of the therapeutic morphogen.

In another invention embodiment, the methods described herein can include administering to the mammal an effective amount of at least one morphogenic protein or an effective fragment thereof directly to or near a wound in need of treatment, either alone or in combination with a nucleic acid as described above.

In another aspect, the invention features a method for inducing new blood vessel growth in the skin of a mammal in need of such treatment. In one embodiment, the method includes administering a therapeutically effective amount of at least one morphogenic protein or effective fragment thereof, or a nucleic acid encoding the morphogen. Typically, such a method further includes selecting a mammal such as a human or veterinary patient having a wound or wounding disorder, and administering the protein or nucleic acid directly to or near a skin wound in need of treatment. Also typically, the method includes expressing the morphogen or fragment in or near the wound in the patient to prevent, treat or reduce the severity of symptoms of the wounding disorder and to enhance or accelerate healing. Any suitable method of administering the nucleic acid, such as those methods already described, can be used. According to the invention method, the skin of the subject can typically be impacted by a wound or wounding disorder, including diabetes, however the method may also be used to increase blood flow in the unwounded skin of patients, for example those with vascular insufficiency, or for cosmetic purposes.

The method may be used alone or in further combination with administration to the skin of the patient at least one nucleic acid encoding at least one of an angiogenic or hematopoietic protein, or effective fragment thereof as described previously. Alternatively, or in addition, the method can further include the step of administering to the mammal an effective amount of at least one morphogenic protein or an effective fragment thereof.

As demonstrated in Examples below, one advantageous effect of topically applying a nucleic acid encoding a morphogen to a wound is the incorporation of bone-marrow derived endothelial progenitor cells (EPCs) into new blood vessels including microvasculature that form in the healing tissue. Accordingly, and in one aspect, the invention provides a method for increasing recruitment of EPCs into vasculature in the skin of a mammal. The method includes contacting the skin of the mammal with an effective amount of at least one morphogenic protein or effective fragment thereof, or a nucleic acid encoding such a protein.

There are reports that new blood vessel growth can be facilitated by administering to a mammal a preparation that includes endothelial cells (ECs), endothelial progenitor cells (EPCs) or both. Thus the present invention further provides for methods in which administration of a nucleic acid encoding the morphogen or effective fragment additionally includes administering a therapeutically effective amount of endothelial cells (ECs) or endothelial cell precursors (EPCs). Preferred ECs and EPCs are characterized by having at least one of the following markers: CD34⁺, flk-1⁺, and tie-2⁺. See, for example, U.S. Pat. Nos. 6,659,428; 5,980,887; EP1061800; WO 99/45775; U.S. Pat. Nos. 5,830,879; 6,258,787; 6,121,246; RE 37,933, 5,851,521 and 5,106,386, (disclosing methods for preparing and using ECs and EPCs to promote blood vessel growth).

In another aspect, the invention features a pharmaceutical product (or “hedgehog therapeutic”) for preventing, treating or reducing the severity of a wound or wounding disorder or accelerating wound healing in a mammal. In one embodiment, the product includes at least one morphogenic protein or effective fragment thereof, or a nucleic acid encoding the morphogen or fragment, formulated to be physiologically acceptable to a mammal and suitable for topical administration. The therapeutic product of the present invention can be formulated as a preparation to be applied topically to a patient's skin, such as in an emulsion, lotion, spray, powder, ointment, cream, or foam or in other suitable pharmaceutical vehicles or carriers commonly known in the art. Preferably, the product is sterile and optionally, the product further includes at least one of an angiogenic or hematopoietic protein, or a nucleic acid encoding the protein. If desired, the pharmaceutical product can further include endothelial cells (ECs), endothelial progenitor cells (EPCs) or both cell types.

The source of the hedgehog therapeutics to be formulated will depend on the particular form of the agent. Peptidyl fragments can be chemically synthesized and provided in a pure form suitable for pharmaceutical/cosmetic usage. Products of natural extracts can be purified according to techniques known in the art. For example, the Cox et al. U.S. Pat. No. 5,286,654 describes a method for purifying naturally occurring forms of a secreted protein and can be adapted for purification of hedgehog polypeptides. Recombinant sources of hedgehog polypeptides are also available, as discussed above.

Those of skill in treating disorders and lesions of cutaneous, mucosal, and vascular surfaces can determine the effective amount of a hedgehog therapeutic to be formulated in a pharmaceutical or cosmetic preparation.

The hedgehog therapeutic formulations of the invention are most preferably applied in the form of appropriate compositions. Appropriate compositions are all compositions usually employed for topically or systemically administering drugs. The pharmaceutically acceptable carrier should be substantially inert, so as not to act with the active component. “Pharmaceutically acceptable carriers” for therapeutic use are well known in the pharmaceutical arts, and are described, for example, in Remington's Pharmaceutical Sciences, Mack Publishing Co. (A. R. Gennaro, editor, 1985). Suitable inert carriers include water, alcohol polyethylene glycol, mineral oil or petroleum gel, propylene glycol and the like. One preferred carrier for use with nucleic acids is methylcellulose.

To prepare the pharmaceutical compositions of this invention, an effective amount of the particular hedgehog therapeutic as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, particularly, for administration percutaneously (or transdermally), to a mucosal surface, orally, rectally, or by parenteral injection. For example, in the compositions suitable for percutaneous or transmucosal administration, the carrier optionally comprises a penetration enhancing agent and/or a suitable wetting agent, optionally combined with suitable additives of any nature in minor proportions, which additives do not introduce a significant deleterious effect on the skin or mucosal surface (such as the oral mucosa).

Also included are solid form preparations which are intended to be converted, shortly before use, to liquid form preparations. In addition to the direct topical application of the preparations they can be topically administered by other methods, for example, encapsulated in a temperature and/or pressure sensitive matrix or in film or solid carrier which is soluble in body fluids and the like for subsequent release, preferably sustained-release of the active component.

As appropriate compositions for topical application there may be cited all compositions usually employed for topically administering therapeutics, for example creams, jellies, dressings, shampoos, tinctures, pastes, ointments, salves, powders, liquid or semiliquid formulations and the like. Application of the compositions may be by aerosol for example with a propellent such as nitrogen carbon dioxide, a freon, or without a propellent such as a pump spray, drops, lotions, or a semisolid such as a thickened composition which can be applied by a swab. In particular compositions, semisolid compositions such as salves, creams, pastes, jellies, ointments and the like will conveniently be used. The compositions can be incorporated into a patch, such as a transdermal patch. The compositions can also be formulated in a collagen matrix such as artificial skin.

As used herein, the expressions “application to the skin,” “topical application,” and “application to a body surface” are intended to encompass application of the composition to either an intact body surface or to wounded body surface. For example, topical application to intact skin (for example, for a cosmetic purpose) would involve application to the epidermis, whereas topical application to a third degree burn would involve application to deeper subepidermal and even dermal tissues exposed to the surface after the burn. Similarly, application to a body surface such as the gastrointestinal mucosa for treatment of a lesion such as an ulcer includes application to any cellular layer comprising the mucosa, intact or damaged.

The pharmaceutical preparations of the present invention can be used, as stated above, for the many applications which can be considered cosmetic uses. Cosmetic compositions known in the art, preferably hypoallergic and pH controlled, are especially preferred, and include toilet waters, packs, lotions, skin milks or milky lotions. The preparations contain, besides the hedgehog therapeutic, components usually employed in such preparations. Examples of such components are oils, fats, waxes, surfactants, humectants, thickening agents, antioxidants, viscosity stabilizers, chelating agents, buffers, preservatives, perfumes, dyestuffs, lower alkanols, and the like. If desired, further ingredients may be incorporated in the compositions, for example, anti-inflammatory agents, antibacterials, antifungals, disinfectants, vitamins, sunscreens, antibiotics, or anti-acne agents.

Examples of oils include fats and oils such as olive oil and hydrogenated oils, waxes such as beeswax and lanolin, hydrocarbons such as liquid paraffin, ceresin, and squalane, fatty acids such as stearic acid and oleic acid, alcohols such as cetyl alcohol, stearyl alcohol, lanolin alcohol, and hexadecanol, and esters such as isopropyl myristate, isopropyl palmitate and butyl stearate. Examples of surfactants include anionic surfactants such as sodium stearate, sodium cetylsulfate, polyoxyethylene laurylether phosphate, sodium N-acyl glutamate, cationic surfactants such as stearyldimethylbenzylammonium chloride and stearyltrimethylammonium chloride, ampholytic surfactants such as alkylaminoethylglycine hydrocloride solutions and lecithin, and nonionic surfactants such as glycerin monostearate, sorbitan monostearate, sucrose fatty acid esters, propylene glycol monostearate, polyoxyethylene oleylether, polyethylene glycol monostearate, polyoxyethylene sorbitan monopalmitate, polyoxyethylene coconut fatty acid monoethanolamide, polyoxypropylene glycol, polyoxyethylene, castor oil, and polyoxyethylene lanolin. Examples of humectants include glycerin, 1,3-butylene glycol, and propylene glycol; examples of lower alcohols include ethanol and isopropanol; examples of thickening agents include xanthan gum, hydroxypropyl cellulose, hydroxypropyl methyl cellulose, polyethylene glycol and sodium carboxymethyl cellulose; examples of antioxidants comprise butylated hydroxytoluene, butylated hydroxyanisole, propyl gallate, citric acid and ethoxyquin. Examples of chelating agents include disodium edetate and ethanehydroxy diphosphate, examples of buffers comprise citric acid, sodium citrate, boric acid, borax, and disodium hydrogen phosphate, and examples of preservatives are methyl parahydroxybenzoate, ethyl parahydroxybenzoate, dehydroacetic acid, salicylic acid and benzoic acid.

For preparing ointments, creams, toilet waters, skin milks, and the like, typically from 0.01 to 10%, preferably from 0.1 to 5% and more preferably from 0.2 to 2.5% of the active ingredient, for example of the hedgehog therapeutic is incorporated in the compositions. In ointments or creams, the carrier for example consists of 1 to 20%, preferably 5 to 15% of a humectant; 0.1 to 10% and preferably from 0.5 to 5% of a thickener and water; or the carrier may consist of 70 to 99%, and preferably 20 to 95% of a surfactant, and 0 to 20%, and preferably 2.5 to 15% of a fat; or 80 to 99.9% and preferably 90 to 99% of a thickener; or 5 to 15% of a surfactant, 2-15% of a humectant, 0 to 80% of an oil, very small (<2%) amounts of preservative, coloring agent and/or perfume, and water. In a toilet water, the carrier for example consists of 2 to 10% of a lower alcohol, 0.1 to 10% and preferably 0.5 to 1% of a surfactant, 1 to 20%, and preferably 3 to 7% of a humectant, 0 to 5% of a buffer, water and small amounts (<2%) of preservative, dyestuff and/or perfume. In a skin milk, the carrier typically consists of 10-50% of oil, 1 to 10% of surfactant, 50-80% of water and 0 to 3% of preservative and/or perfume. In the aforementioned preparations, all % symbols refer to weight by weight percentage.

Particular compositions for use in the method of the present invention are those wherein the hedgehog therapeutic is formulated in liposome-containing compositions. Liposomes are artificial vesicles formed by amphiphatic molecules such as polar lipids, for example, phosphatidyl cholines, ethanolamines and serines, sphingomyelins, cardiolipins, plasmalogens, phosphatidic acids and cerebiosides. Liposomes are formed when suitable amphiphathic molecules are allowed to swell in water or aqueous solutions to form liquid crystals usually of multilayer structure comprised of many bilayers separated from each other by aqueous material (also referred to as coarse liposomes). Another type of liposome known to be consisting of a single bilayer encapsulating aqueous material is referred to as a unilamellar vesicle. If water-soluble materials are included in the aqueous phase during the swelling of the lipids they become entrapped in the aqueous layer between the lipid bilayers.

Water-soluble active ingredients such as, for example, various salt forms of a hedgehog polypeptide, are encapsulated in the aqueous spaces between the molecular layers. The lipid soluble active ingredient of hedgehog therapeutic, such as an organic mimetic, is predominantly incorporated into the lipid layers, although polar head groups may protrude from the layer into the aqueous space. The encapsulation of these compounds can be achieved by a number of methods. The method most commonly used involves casting a thin film of phospholipid onto the walls of a flask by evaporation from an organic solvent. When this film is dispersed in a suitable aqueous medium, multilamellar liposomes are formed. Upon suitable sonication, the coarse liposomes form smaller similarly closed vesicles.

Water-soluble active ingredients are usually incorporated by dispersing the cast film with an aqueous solution of the compound. The unencapsulated compound is then removed by centrifugation, chromatography, dialysis or other suitable procedures known in the art. The lipid-soluble active ingredient is usually incorporated by dissolving it in the organic solvent with the phospholipid prior to casting the film. If the solubility of the material in the lipid phase is not exceeded or the amount present is not in excess of that which can be bound to the lipid, liposomes prepared by the above method usually contain most of the material bound in the lipid bilayers; separation of the liposomes from unencapsulated material is not required.

A particularly convenient method for preparing liposome formulated forms of hedgehog therapeutics is the method described in EP-A-253,619, incorporated herein by reference. In this method, single bilayered liposomes containing encapsulated active ingredients are prepared by dissolving the lipid component in an organic medium, injecting the organic solution of the lipid component under pressure into an aqueous component while simultaneously mixing the organic and aqueous components with a high speed homogenizer or mixing means, whereupon the liposomes are formed spontaneously.

The single bilayered liposomes containing the encapsulated hedgehog therapeutic can be employed directly or they can be employed in a suitable pharmaceutically acceptable carrier for topical administration. The viscosity of the liposomes can be increased by the addition of one or more suitable thickening agents such as, for example xanthan gum, hydroxypropyl cellulose, hydroxypropyl methylcellulose and mixtures thereof. The aqueous component may consist of water alone or it may contain electrolytes, buffered systems and other ingredients, such as, for example, preservatives. Suitable electrolytes which can be employed include metal salts such as alkali metal and alkaline earth metal salts. The preferred metal salts are calcium chloride, sodium chloride and potassium chloride. The concentration of the electrolyte may vary from zero to 260 mM, preferably from 5 mM to 160 mM. The aqueous component is placed in a suitable vessel which can be adapted to effect homogenization by effecting great turbulence during the injection of the organic component. Homogenization of the two components can be accomplished within the vessel, or, alternatively, the aqueous and organic components may be injected separately into a mixing means which is located outside the vessel. In the latter case, the liposomes are formed in the mixing means and then transferred to another vessel for collection purposes.

The organic component consists of a suitable non-toxic, pharmaceutically acceptable solvent such as, for example ethanol, glycerol, propylene glycol and polyethylene glycol, and a suitable phospholipid which is soluble in the solvent. Suitable phospholipids which can be employed include lecithin, phosphatidylcholine, phosphatydylserine, phosphatidylethanol-amine, phosphatidylinositol, lysophosphatidylcholine and phospha-tidyl glycerol, for example. Other lipophilic additives may be employed in order to selectively modify the characteristics of the liposomes. Examples of such other additives include stearylamine, phosphatidic acid, tocopherol, cholesterol and lanolin extracts.

In addition, other ingredients which can prevent oxidation of the phospholipids may be added to the organic component. Examples of such other ingredients include tocopherol, butylated hydroxyanisole, butylated hydroxytoluene, ascorbyl palmitate and ascorbyl oleate. Preservatives such a benzoic acid, methyl paraben and propyl paraben may also be added.

Apart from the above-described compositions, use may be made of covers, for example plasters, bandages, dressings, gauze pads, transdermal patches and the like, containing an appropriate amount of a hedgehog therapeutic. In some cases use may be made of artificial skin which has been impregnated with a topical formulation containing the therapeutic formulation.

In preparing the compositions in oral dosage form, any of the usual pharmaceutical media may be employed such as, for example, water, glycols, oils, alcohols and the like in the case of oral liquid preparations such as suspensions, syrups, elixirs and solutions, or solid carriers such as starches, sugars, kaolin, lubricants, binders, disintegrating agents and the like in the case of powders, pills, capsules, and tablets. Because of their ease in administration, tablets and capsules represent the most advantageous oral dosage unit form, in which case solid pharmaceutical carriers are employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed.

It is especially advantageous to formulate the subject compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powders packets, wafers, injectable solutions or suspensions, teaspoonfuls, tablespoonfuls and the like, and segregated multiples thereof.

Further provided by the invention is a kit for the introduction of at least one morphogen into the skin of a mammal. Preferred kits include at least one morphogen or effective fragment thereof, or a nucleic acid encoding at least one morphogen or fragment (such as an N-terminal fragment), and optionally at least one angiogenic or hematopoietic protein or nucleic acid encoding same. In one embodiment, the kit further includes a pharmacologically acceptable carrier solution, and means for delivering at least the morphogen to the mammal and directions for using the kit. If desired, the pharmaceutical product can further include endothelial cells (ECs), endothelial progenitor cells (EPCs) or both cell types. Preferably in such embodiments, the kit further includes means for topical delivery of the product to the mammal such as a syringe, spray mechanism, or related device, or the kit can include a patch such as a transdermal patch formulated to adhere to a body surface (typically the skin) and to release the therapeutic during wear by the user.

It has been further discovered that another beneficial effect of topical application of a morphogen to the skin is likely to be an increase in the production of cytokines and angiogenic factors by cells of the skin. Studies performed on cultured fibroblasts from the dermis of the skin, and EPCs, described in Examples below, show that these cells of the skin respond to application of morphogens such as human Shh protein by upregulating expression of several cytokines and factors important for angiogenesis and intercellular communication, including Glc-1, Ptc-1, VEGF, angiopoietin 1 and SDF-1 alpha. Accordingly, yet another aspect of the invention is a method for increasing production of at least one cytokine by cells of the skin, comprising contacting the cells with an effective amount of at least one morphogenic protein or effective fragment thereof, or a nucleic acid encoding the morphogen. As discussed, the cytokines can include but are not limited to Glc-1, Ptc-1, VEGF, angiopoietin 1 and SDF-1 alpha.

In some applications it may be desirable to treat skin cells with the morphogen in vitro, either as explants or skin grafts, or as dissociated cells. Depending on the purpose, suitable tissue or cells can be obtained as explants or primary cultures, or as cell lines obtained, for example, from the American Type Tissue Collection (Manassas, Va.). The method may also be performed by applying the morphogen to normal or wounded skin of a subject, for example to increase the production of these factors in the skin of a patient in need of such treatment.

From other studies (see Examples below) it has been demonstrated that yet another beneficial effect of morphogen administration to skin cells is promotion of cell proliferation by at least one cell type of the skin tested, i.e., fibroblasts of the dermis, as well as EPCs. It is believed that stimulation of proliferation of these cells can promote wound healing by contributing substantially to the thickness of the new tissue formed during wound repair. Accordingly, the invention further provides a method for increasing cellular proliferation in the skin of a mammal. The method includes contacting skin cells with an effective amount of at least one morphogenic protein or effective fragment thereof, or a nucleic acid encoding same. As in the method for increasing production of cytokines by skin cells, variations of the method can be practiced both in vitro and in vivo.

### EXAMPLES

The following Examples are put forth to provide those of ordinary skill in the art with a complete disclosure and description of how to make and use the present invention, and are not intended to limit the scope of what the inventors regard as their invention. Neither are they intended to represent that the experiments below are all or the only experiments performed. Efforts have been made to ensure accuracy with respect to numbers used (for example, amounts, temperature, etc.), but some experimental errors and deviations should be accounted for. Unless indicated otherwise, parts are parts by weight, molecular weight is weight average molecular weight, and temperature is in degrees Celsius

Examples 1-8 below describe materials and methods that may be used in the practice of the invention and/or are demonstrated in studies described in Examples 9 and higher, infra.

### Example 1

### Preparation of Human Shh Plasmid

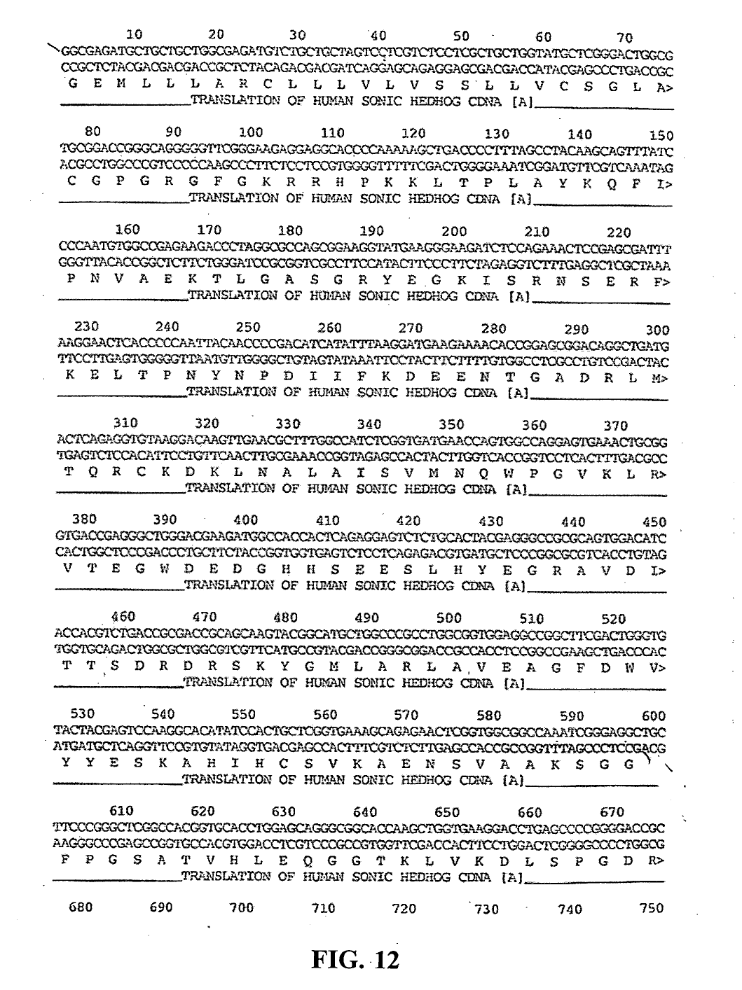

The amino-terminal domain of human Shh was selected as coding sequence to make a Shh-plasmid using mammalian expression vector pCMV-ScriptPCR (Stratagene). The selected sequence of the Shh cDNA is shown schematically in FIG. 12. This plasmid of human Shh (phShh) is a 4,878-bp plasmid that contains the 600 bp amino terminal domain coding sequence of human Shh. Expression of the Shh gene is modulated by the presence of cytomegalovirus promoter sequences. Downstream from the Shh cDNA is an SV40 polyadenylation sequence. The plasmid also contains a gene that confers neomycin/kanamycin resistance to the host cells.

### Example 2

### Experimental Animals

C57BLKS/J-m+/+Lepr^{db} mice (db/db mice), C57BLKS/J (wild-type of db/db mice), GFP-transgenic (Tg) mice, and C57BL/6 (wild-type of GFP Tg mice) were obtained from Jackson Laboratories (Bar Harbor, Me., USA). Animals of the db/db strain are leptin receptor-deficient diabetic mice, and are an established model of deficient wound healing associated with diabetes [14]. NLS-Ptc1-lacZ mice or their wild type littermates were kindly provided by Dr. MP Scott (Stanford University). We also used BMT mice created by transplantation of BM from GFP transgenic mice. BMT mice were prepared as previously described with minor modifications [15, 16]. BM cells were collected from femurs and tibias of donor GFP Tg or wild-type B6 mice by aspiration and flushing. Recipient mice were lethally irradiated with 12.0 Gy, and BMT from the transgenic mice was performed. At 4 weeks after BMT, by which time the bone marrow of recipient mice was reconstituted with the transplanted bone marrow, skin wounds were made on the recipient mice, as described below. All procedures were performed in accordance with the Institutional Animal Care and Use Committee of St. Elizabeth's Medical Center.

Studies were carried out using the above strains of mice to test the effects of phShh topical gene therapy on wound healing in the skin. In some experiments, the expression of the hedgehog receptor Ptc 1 was examined. β-galactosidase (β-gal) staining of wound tissues was analyzed in Ptc1-lacZ mice that carry a mutation of one allele of the Ptc1 gene consisting of insertion of a lacZ reporter gene upstream of the ptch coding region (Goodrich et al. 1997). Male or female NLS-Ptc1-lacZ mice, or their wild type littermates were used.

Because poor capacity for wound healing is associated with diabetes, some investigations were conducted using a genetically diabetic (db) strain of mice. Other studies aimed at determining the origin of cells that appear in healing skin wounds following Shh therapy used bone marrow transplant (BMT) animal models, for example chimeric tie-2/LacZ/BMT mice. These mice are the recipients of bone marrow from donors in which cells express lacZ under control of the endothelial specific Tie-2 promoter. Accordingly, cells of endothelial lineage that are derived from a bone marrow progenitor cell can be identified in these mice by virtue of their lacZ expression, which can be detected by specific staining. After recovery from the bone marrow transplantation, these mice were subjected to a wound healing assay as described below, and were randomly assigned to treatment with phShh plasmid or control plasmid (empty vector) and were sacrificed at selected times thereafter. Similar studies were carried out using GFP/BMT mice.

### Example 3

### Topical Application of phShh and Methods for Evaluation of Wound Healing

DNA/methylcellulose pellets were prepared, as described previously [17]. Briefly, One hundred μg of phShh or LacZ plasmid was diluted in ddH₂O (20 μl) and mixed with an equal volume of 1% methylcellulose prepared in ddH₂O. This solution was then allowed to dry, forming a pellet. Immediately after wounding, the dehydrated pellet containing plasmid was applied to the wound.

All mice used in the wound healing assays were between 8 and 12 weeks of age at time of wounding. Mice were placed individual cages and subjected to wounding, performed as described previously [18]. After induction of deep anesthesia by intraperitoneal injection of sodium pentobarbital (160 mg/kg IP), full-thickness excisional skin wounds were made using 8-mm skin biopsy punches on the backs of mice. Immediately after wounding, a methylcellulose pellet containing phShh or control lacZ plasmid was applied to the wound. The wound was then covered with a semipermeable polyurethane dressing OpSite® (Smith and Nephew, Massillon, Ohio). Opsite®, skin and muscle surrounding wound were sutured together using 6-0 prolene to prevent the mouse from removing the dressing.

In some wounding experiments, wild type and genetically altered mice as described received full thickness excisional wounds as described. Plasmid DNA (either phShh or control LacZ plasmid) was administered directly to the wound, and the wounded areas were harvested at various times, i.e., 1, 4, 5, 10, and 14 days after wounding.

For serial analysis of wound closure, a total of 5 db/db mice were used at each time point. Wound closure was documented with a digital camera (Nikon Coolpix 995, Nikon, Japan) on day 0, 5, 10, and 14. Images were analyzed using the NIH Image J analyzer by tracing the wound margin with a fine resolution computer mouse and calculating pixel area. The areas of the wounds were compared using the paired Student's t-test. For histological scoring, wounds were harvested 10 days after application of either control (lacZ) or phShh. Histological scores were assigned in a blinded manner, as described [18].

The tissue was subjected to several analyses (described below) at various times after wounding, including evaluation of expression of hShh protein and mRNA, measurement of the wound area, histological assessment, and determination of vascular density. Histological assessment of the wound tissues was carried out in fixed sections stained with hematoxylin and eosin (H&E) or Masson's trichrome (MT).

The measurement of wound area was used to calculate the percentage of wound closure after treatment with phShh or control plasmid. Wounds were measured at day 0 and on selected days after wounding. The percentage of wound closure was determined according to the following formula:

$$\% wound closure = \frac{open area on day 0 - open area on final day}{open area on day 0} \times 100$$

Vascular density analysis was performed as described below.

Fluorescence microscopic evaluation of wound vascularity. Fourteen days after creation of wounds and application of either PhShh or lacZ plasmid, animals were prepared for vascular labeling with Rhodamine-conjugated BS1 lectin (Sigma). Before sacrifice, 75 μl of BS1 lectin was injected into the left ventricle to visualize functional vasculature in the healing wound. The lectin was allowed to perfuse for 10 minutes in the animal. After 10 minutes, the chest was entered, the left ventricle was cannulated and the right ventricle was incised. The animal was perfused with phosphate-buffered saline and fixed with 4% paraformaldehyde. The wounds were then harvested from the dorsum of the animals. Vascularity was analyzed as described previously [19].

Immunofluorescence and immunohistochemistry. Immunohistochemistry for Shh was performed on 4% paraformaldehyde-fixed paraffin-embedded tissues sections (5-μm thick). Tissues were blocked with 3% hydrogen peroxide and 5% goat serum, and treated with a 1:200 dilution of rabbit polyclonal anti-Shh antibody (Santa Cruz Biotech, Santa Cruz, Calif.) followed by a biotinylated goat anti-rabbit antibody and Vectastain ABC reagent (Vector Laboratories. Burlingame. Calif.). Multi-color immunofluorescence of GFP/BMT mice was performed on frozen sections (6-μm thick).

Cell culture. Fibroblasts were isolated from wild-type C57BL by placing explants (1×1 mm) on plastic culture dishes that were subsequently cultured in Dulbecco's modified Eagle's medium containing 10% fetal bovine serum after the tissue had dried to the plate. Tissue explants were grown to confluency and passages as needed.

Ex vivo expansion of EPC was performed as described [23]. In brief, BM cells obtained by flushing the tibias and femurs were plated on rat plasma vitronectin-coated (Sigma) culture dishes and maintained in EC basal medium-2 (EBM-2) (Clonetics), supplemented with 5% fetal bovine serum, human VEGF-A, human fibroblast growth factor-2, human epidermal growth factor, insulin-like growth factor-1, and ascorbic acid. After 4 days in culture, non-adherent cells were removed by washing, new media was applied, and the culture was maintained through day 7.

X-gal staining. Skin tissues from NLS-Ptc1-lacZ mice were harvested and processed as described previously [22]. Histological sections were counterstained with nuclear fast red.

Labeling of functional vessels. This procedure was performed by injection of Rhodamine-conjugated BS1 lectin before sacrifice, as described above. Cultured endothelial precursor cells (EPCs) were co-stained with acetylated LDL (acLDL)-DiI (Biomedical Technologies) and FITC-conjugated isolectin B4 (Vector Laboratories), both of which are features characteristic of endothelial lineage [20,21]. Rabbit anti-galactosidase antibody (Cortex) was used to detect lacZ expression in EPCs derived from NLS-Ptc1-lacZ mice. Blue fluorescence was generated with AMCA streptavidin (Vector Laboratories) and biotinylated anti-rabbit antibody (Signet).

### Example 4

### Quantitative RT-PCR of Gli1, cytokines and SDF-1α

The expression of the Hh related transcriptional factor Gli-1 and certain angiogenic cytokines (i.e., VEGF-1, angiopoietin-1 and 2), as well as trafficking chemokine for hematopoietic stem cells (SDF-1α) was evaluated in treated (phShh) and control (pLacZ) fibroblasts.

For in vivo studies, skin samples were harvested 4 days after surgery and homogenized in RNA-Stat (Tel-Test Inc.). RNA was isolated according to the manufacturer's instructions.

For in vitro studies, wild-type fibroblasts were plated in non-coating 35 mm plates at a density of 10,000 cells/well in DMEM containing 10% FBS for 24 h. Wild-type EPCs were plated in non-coating 35 mm plates at a density of 4×10⁵ cells/well in EBM-2 containing 5% FBS for 24 hours. Oct-Shh protein (hydrophobic modified protein designed to increase its activity [24] was supplemented at the appropriate concentration (0, 0.5, 1, and 5 μg/ml) in serum free culture medium. Cells were harvested after 24 hours and RNA was extracted using RNA-Stat™ according to the manufacturer's protocol. Total RNA was reverse transcribed using iScript cDNA Synthesis Kit (Bio Rad) and amplification was performed on the Taqman 7300 (Applied Biosystems).

The PCR conditions were as follows: hold for 2 min at 50° C., and 10 min at 95° C. followed by 2 step PCR for 40 cycles of 95° C. for 15 seconds and 60° C. for 60 seconds. Each sample contained 1 μl cDNA in a 20 μl total reaction using Platinum Quantitative PCR Supermix-UDG (Invitrogen).

Primer and probe sequences were as follows:

| | | | |
|---|---|---|---|
| Shh: | forward | 5′-GAGCAGACCGGCTGATGACT-3′ | (SEQ ID NO:4) |
| | reverse | 5′-AGAGATGGCCAAGGCATTTAAC-3′ | (SEQ ID NO:5) |
| | and | | |
| | | FAM-AGAGGTGCAAAGACA-MGB. | (SEQ ID NO:6) |
| Dhh: | forward | 5′-CGCAGACCGCCTGATGAC-3′ | (SEQ ID NO:7) |
| | reverse | 5′-GCGATGGCTAGAGCGTTGAC-3′ | (SEQ ID NO:8) |
| | and | | |
| | | FAM-AGCGTTGCAAAGAG-MGB. | (SEQ ID NO:9) |
| Ihh: | forward | 5′-CAAACCGGCTGAGAGCTTTC-3′ | (SEQ ID NO:10) |
| | reverse | 5′-AGCCGACGCGGAGGAT-3′ | (SEQ ID NO:11) |
| | and | | |
| | | FAM-AGGTCATCGAGACTCA-MGB. | (SEQ ID NO:12) |
| Gli-1: | forward | 5′-CGTCACTACCTGGCCTCACA-3′ | (SEQ ID NO:13) |
| | reverse | 5′-CCCCCTGGCTGAAGCATAT-3′ | (SEQ ID NO:14) |
| | and | | |
| | | FAM-CCAGCACTACATGCTCCGGGCAA-TAMRA. | (SEQ ID NO:15) |
| VEGF: | forward | 5′-CAAAAACGAAAGCGCAAGAAA-3′ | (SEQ ID NO:16) |
| | reverse | 5′-CGCTCTGAACAAGGCTCACA-3′ | (SEQ ID NO:17) |
| | and | | |
| | | FAM-CCCGGTTTAAATCCTGGAGCGTTCA-TAMRA. | (SEQ ID NO:18) |
| Angio- | forward | 5′-CAGATACAACAGAATGCGGTTCA-3′ | (SEQ ID NO:19) |
| poietin-1: | reverse | 5′-TGAGACAAGAGGCTGGTTCCTAT-3′ | (SEQ ID NO:20) |
| | and | | |
| | | FAM-AACCACACGGCCACCATGCTGG-TAMRA. | (SEQ ID NO:21) |
| Angio | forward | 5′-CTACAGGATTCACCTTACAGGACTCA-3 | (SEQ ID NO:22) |
| poietin-2: | reverse | 5′-CTTCCTGGTTGGCTGATGCT-3′ | (SEQ ID NO:23) |
| | and | | |
| | | FAM-TGATTTTGCCCGCCGTGCCT-TAMRA. | (SEQ ID NO:24) |
| IGF-1: | forward | 5′-CCTACAAAGTCAGCTCGTTCCA-3′ | (SEQ ID NO:25) |
| | reverse | 5′-TCCTTCTGAGTCTTGGGCATGT-3′ | (SEQ ID NO:26) |
| | and | | |
| | | FAM-CGGGCCCAGCGCCACACT-TAMRA. | (SEQ ID NO:27) |
| SDF-1α: | forward | 5′-ATCAGTTACGGTAAGCCAGTCA-3′ | (SEQ ID NO:28) |
| | reverse | 5′-TGGCGACATGGCTCTCAAA-3′ | (SEQ ID NO:29) |
| | and | | |
| | | FAM-CTGAGCTACAGATGCCCCTGCCGATT-TAMRA. | (SEQ ID NO:30) |

In some experiments, (see, e.g., Examples 9, 10, and 15) other primers were used as follows:

| | | | |
|---|---|---|---|
| Shh: | forward | 5′-AAGGACAAGTTGAACGCTTTGG-3′, | (SEQ ID NO:31) |
| | reverse | 5′-TCGGTCACCCGCAGTTTC-3′, | (SEQ ID NO:32) |
| | and | | |
| | | FAM-CTCCTGGCCACTGGTTCATCACCG-TAMRA. | (SEQ ID NO:33) |
| Gli1: | forward | 5′-CACCACCCTACCTCTGTCTATTCG-3′, | (SEQ ID NO:34) |
| | reverse | 5′-TCCTGTAGCCCCCTAGTATCCA-3′, | (SEQ ID NO:35) |
| | and | | |
| | | FAM-CCCAGCATCACCGAAAATGTTGCC-BHQ, | (SEQ ID NO:36) |
| PTC1: | forward | 5′-CTCTGGAGCAGATTTCCAAGG-3′, | (SEQ ID NO:37) |
| | reverse | 5′-TGCCGCAGTTCTTTTGAATG-3′, | (SEQ ID NO:38) |
| | and | | |
| | | FAM-AAGGCTACTGGCCGGAAAGCGC-TAMRA. | (SEQ ID NO:39) |
| VEGF: | forward | 5′-CATCTTCAAGCCGTCCTGTGT-3′, | (SEQ ID NO:40) |
| | reverse | 5′-CAGGGCTTCATCGTTACAGCA-3′, | (SEQ ID NO:41) |
| | and | | |
| | | FAM-CCGCTGATGCGCTGTGCAGG-BHQ. | (SEQ ID NO:42) |
| Angio- | forward | 5′-GGGACAGCAGGCAAACAGA-3′, | (SEQ ID NO:43) |
| poietin-1: | reverse | 5′-TGTCGTTATCAGCATCCTTCGT-3′, | (SEQ ID NO:44) |
| | and | | |
| | | FAM-TTGATCTTACACGGTGCCGATT-BHQ. | (SEQ ID NO:45) |
| Angio- | forward | 5′-TCAGCCAACCAGGAAGTGATT-3′, | (SEQ ID NO:46) |
| poietin-2: | reverse | 5′-AGCATCTGGGAACACTTGCAG-3′, | (SEQ ID NO:47) |
| | and | | |
| | | FAM-CACAAAGGATTCGGACAATGACAAATGCA-BHQ. | (SEQ ID NO:48) |
| SDF-1.: | forward | 5′-CCTCCAAACGCATGCTTCA-3′, | (SEQ ID NO:49) |
| | reverse | 5′-CCTTCCATTGCAGCATTGGT-3′, | (SEQ ID NO:50) |
| | and | | |
| | | FAM-CTGACTTCCGCTTCTCACCTCTGTAGCCT-TAMRA. | (SEQ ID NO:51) |
| 18S: | forward | 5′-CGGGTCGGGAGTGGGT-3′, | (SEQ ID NO:52) |
| | reverse | 5′-GAAACGGCTACCACATCCAAG-3′, | (SEQ ID NO:53) |
| | and | | |
| | | FAM-TTTGCGCGCCTGCTGCCTT-BHQ. | (SEQ ID NO:54) |

Relative mRNA expression of target genes was calculated with the comparative C_{T} method. The amount of target genes was normalized to the endogenous 18S control gene (Applied Biosystems). Difference in C_{T} values was calculated for each mRNA by taking the mean C_{T} of duplicate reactions and subtracting the mean C_{T} of duplicate reactions for 18S RNA. We calculated the fold change in expression of the target gene from cells treated relative to control cells: relative expression=2^{ΔCT}

### Example 5

### Western Blot Analysis

Protein extracts were prepared using standard techniques from normal and wounded skin samples taken at various times after wounding and topical application of DNA-containing plasmids. For some studies, skin samples were harvested 4 days after surgery and homogenized in lysis buffer and protein extracts were used for Western blotting analysis of Shh. Proteins were detected using primary antibody, rabbit polyclonal against Shh (Santa Cruz Biotech, Santa Cruz, Calif.). Protein samples were used for Western blot analysis of hShh and actin detection with appropriate primary antibodies.

Briefly, total protein extracts were electrophoresed on a 7.5% SDS-polyacrylamide gel and electrophoretically transferred to an Immobilon PVDF membrane (Millipore). Protein standards (BioRad) were run on each gel. The blots were blocked with 5% milk in Tris-buffered saline Tween-20 for 1 hour at room temperature. Blots were incubated overnight at 4° C. with primary antibody and after stringent washing, blots were incubated for 1 hour at room temperature with 1:5000 diluted horseradish peroxidase-conjugated anti-rabbit IgG secondary antibody (Santa Cruz Biotech). Peroxidase activity was visualized by exposing an X-ray film to blots incubated with ECL regent (Amersham). For the loading control, actin was used.

### Example 6

### Immunohistochemical Assessment of Blood Vessels

For visualization of blood vessels in healing wounds, immunohistochemical staining was performed using antibodies prepared against the murine-specific endothelial cell marker isolectin B4 (Vector Laboratories). Capillary density was evaluated morphometrically by histological examination of five randomly selected fields of tissue sections recovered from the wounded areas of skin. Capillaries were recognized as fluorescently labeled tubular structures positive for isolectin B4. All morphometric studies were performed by two examiners who were blinded to treatment.

### Example 7

### Cellular Identification of LacZ-expressing Cells

Tie-2/LacZ/BMT mice were sacrificed several intervals following skin wounding and administration of vectors. Normal skin samples and wounded areas were removed and fixed in 4% paraformaldehyde for 3 hours at room temperature and incubated in X-gal solution overnight at 37° C. The tissue samples were then placed in PBS and examined under a dissecting microscope to detect sites of LacZ-expressing cells macroscopically. In some cases histological sections were counterstained with nuclear fast red under 40× magnification as described. X-gal positive cells (blue stained cells) were counted per sample in a blinded manner.

### Example 8

### Other Assays and Statistical Procedures

Proliferation assay. The proliferative activity of cells treated with Shh was examined using CellTiter 96® nonradioactive cell proliferation Assay (Promega) according to the manufacture's instructions. Briefly, subconfluent cells (fibroblasts: 5000 cells/well, EPCs: 10000 cells/well) were reseeded on 96-well flat-bottomed plates with 100 μl of the growth media. Then cells were treated by Shh (0, 0.5, 1, 5 and 10 μg/ml) and incubated for 48 hours at 37° C. The absorbance at 570 nm wavelength was recorded using a 96 well ELISA plate reader (Bionetics Laboratory).

Migration assay. EPC migration was evaluated using a modified Boyden's chamber assay as described previously [25]. Briefly, a polycarbonate filter (5 μm-pore size) (Poretics) was placed between upper and lower chamber. Cell suspensions (5×10⁴ cells/well) were placed in the upper chamber, and the lower chamber was filled with medium containing human recombinant VEGF (50 ng/ml) (R&D Systems, Minneapolis, Minn., USA) or Shh protein (0, 0.5, 1.0, 5.0, 10.0 μg/ml). The chamber was incubated for 16 h at 37° C. and 5% CO₂. Migration activity was evaluated as the mean number of migrated cells in 5 high power fields (×40) per chamber.

Adhesion assay. After 36 h of incubation with Shh, EPCs were washed with PBS and gently detached with 0.25% trypsin. After centrifugation and resuspension in EBM-2, 5% FBS, identical cell numbers were replated onto vitronectin or laminin-coated culture dishes and incubated for 30 min at 37° C. Adherent cells were counted by independent blinded investigators as described [26].

Tube formation assay. Endothelial tube formation was assessed using Matrigel assay (BD Biosciences). After 24 h of incubation with Shh, EPCs were washed with PBS and gently detached with 0.25% trypsin. Cells were seeded with a density of 3×10⁴/well on 4-well chamber coated with 250 μL Matrigel and incubated with EGM-2 containing 5% FBS for 48 hours at 37° C. Tube formation was examined by a phase-contrast microscopy.

Statistical procedures. All results are expressed as mean±S.E. Statistical significance was evaluated using the unpaired Student's t-test between two means. Multiple comparisons between more than three groups were done by ANOVA. A value of P<0.05 denoted statistical significance. All in vitro experiments were repeated at least in triplicate.

### Example 9

### Hedgehog Signaling Pathway is Activated During Wound Healing

This Example demonstrates that the hedgehog signaling pathway is upregulated in at least one cell type in the skin during wound healing.

The expression of the hedgehog receptor Ptc1 was examined after wounding in wild type mice, and in NLS-ptc1/LacZ mice. As described above, the latter mice carry a mutation of one allele of the Ptc1 gene consisting of insertion of a lacZ reporter gene upstream of the ptch coding region [27].

Briefly, a full thickness excisional skin wound was made on the backs of the animals, and tissue samples from the wounded areas were harvested at 1 and 3 days after wounding to examine for Ptc1 expression, as determined by X-gal staining. As controls, X-gal staining was observed in the normal (unwounded) skin of NLS-ptc1/LacZ mice, and in wild type mice receiving skin wounds.

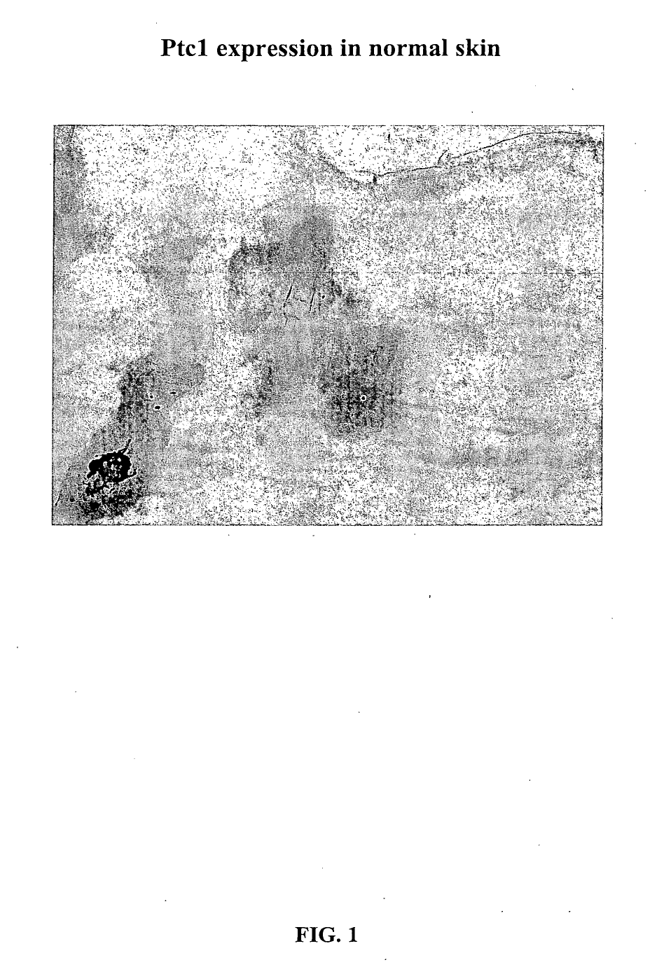

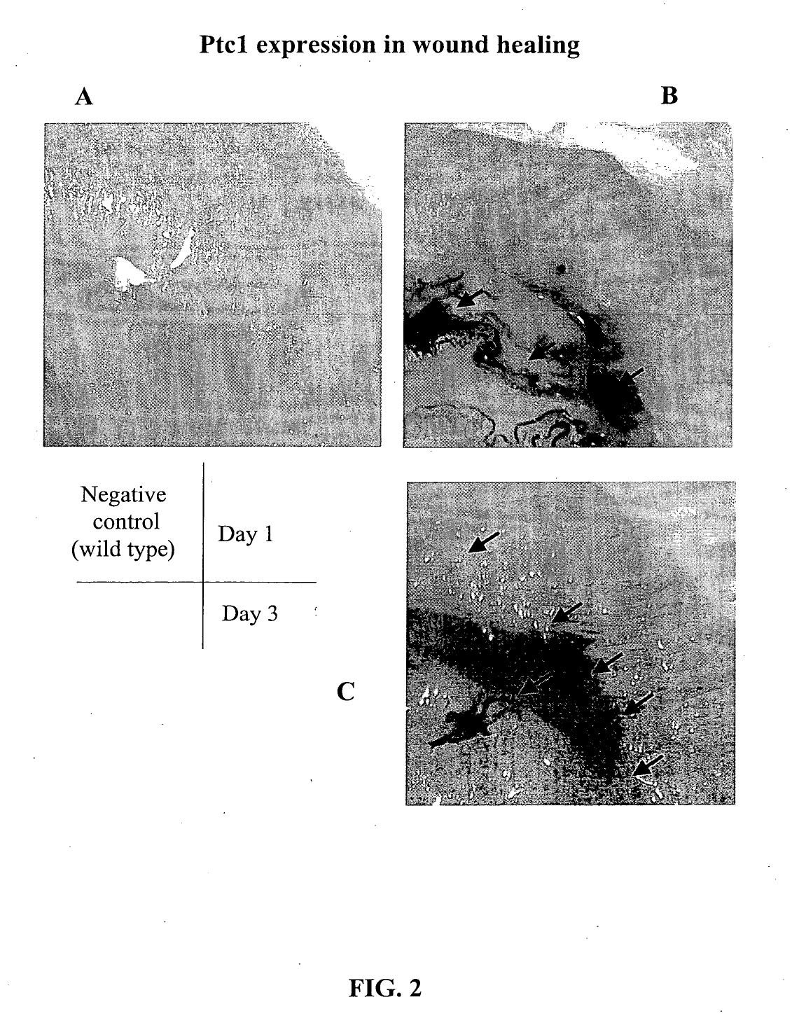

Referring to FIG. 1, in control NLS-ptc1/LacZ mice with normal, unwounded skin, a few X-gal positive cells were localized to hair follicles, and surrounding parts of blood vessels. In the dermal mesenchymal area, only a few cells adjacent to the hair follicles were positive. In contrast, at 1 and 3 days after skin wounding, X-gal positive cells were present not only in hair follicles, but also in the cells in dermis. The Ptc1-positive cells were abundant at the wound edge and the area surrounding vessels. Ptc-1 expression was observed in several types of cells: spindle shaped mesenchymal cells, round shaped infiltrating cells, and microvascular endothelial cells (FIGS. 2B, C). No X-gal positive cells were observed in wounded skin of wild type mice (FIG. 2A).

### Example 10

### Topical Application of Hedgehog Gene to Skin Wounds Promotes Upregulation of Hedgehog mRNA and Protein Expression in Skin Wounds in Normal and Diabetic Mice

As discussed above, wound healing is impaired in several wounding disorders. In particular, healing of wounds in diabetic patients is problematic. This example shows that local gene therapy using a vector expressing human sonic hedgehog (phShh) promotes wound healing in an animal model of diabetes.

For this study, genetically diabetic mice (db strain) received full thickness excisional wounds as described above. DNA in the form of plasmid (either phShh or control LacZ plasmid) was administered directly to the wound, and the wounded areas were harvested at 4, 5, 10, and 14 days after wounding. The tissue was subjected to several analyses (described in Methods above), including evaluation of expression of hShh protein and mRNA at day 4, measurement of the wound area at day 5, 10 and 14, histological assessment at day 10, and determination of vascular density at day 14.

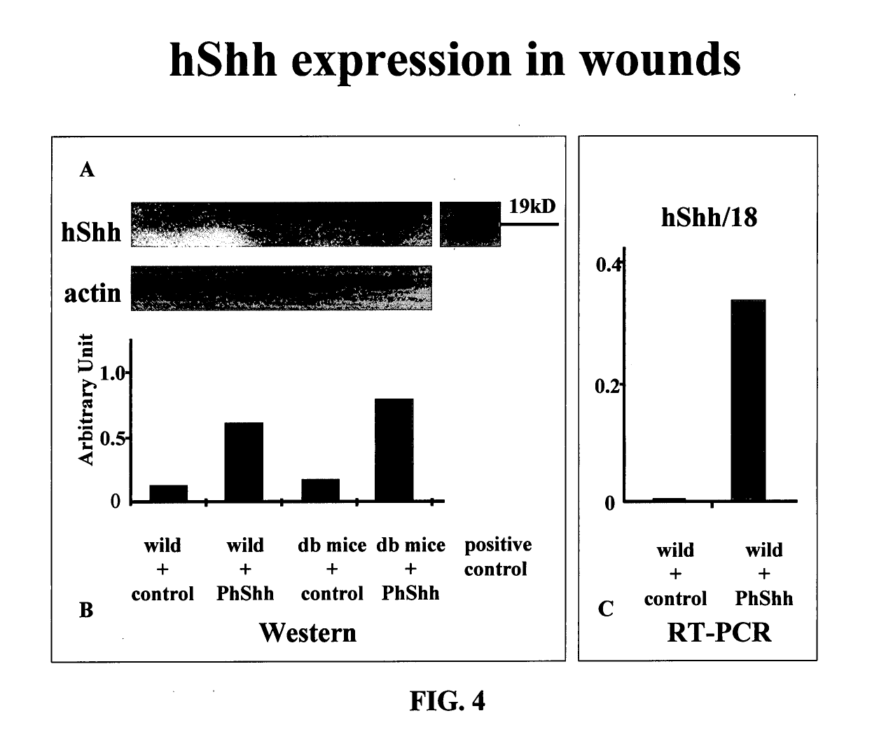

FIGS. 4A and 4B shows the results of Western blot analysis for detection of hShh protein in skin wounds following topical application of phShh or control plasmids to the wounds of wild type mice and diabetic (db) mice. Immunoblots in FIG. 4A show the detection of HShh (upper) and actin (lower). Relative levels of hShh are shown in arbitrary units in FIG. 4B. Referring to FIG. 4B, it was found that in wild type mice, whereas expression of hShh protein was nearly undetectable after application of control plasmid, this protein was clearly detectable following application of PhShh. Even more dramatic upregulation of hShh protein expression was observed in diabetic mice receiving topical application of PhShh (FIG. 4B). The upregulation of hShh mRNA was also confirmed by RT-PCR. Referring to FIG. 4C, a dramatic increase in hShh transcript was observed in skin wounds of animals receiving topical pHShh but not control plasmid.

To confirm that the increased Shh proteins in phShh-treated group detected by Western blotting was not endogenous but derived from phShh, RT-PCR was performed. RT-PCR for human Shh mRNA expression showed that phShh results in a significant increase in human Shh mRNA levels in the wound tissue (FIG. 4C). Furthermore, immunoperoxidase staining for Shh from wounds 4 days after treatment with phShh showed large numbers of positive staining cells at the edge of the wound. In particular, Shh was expressed by a variety of proliferating cells resembling fibroblasts and keratinocytes. In contrast, the wounds in the control group showed no specific staining for Shh.

### Example 11

### Topical Application of Hedgehog Gene to Skin Wounds Hastens Wound Closure in Diabetic Wounds

Animals (diabetic mice) were subjected to the skin wounding assay described in Methods above, with topical application of phShh or control plasmid. At various intervals after wounding (i.e., 5, 10 and 14 days), open wound areas were measured and the percentage of wound closure was determined according to the formula described in Methods above for each condition.

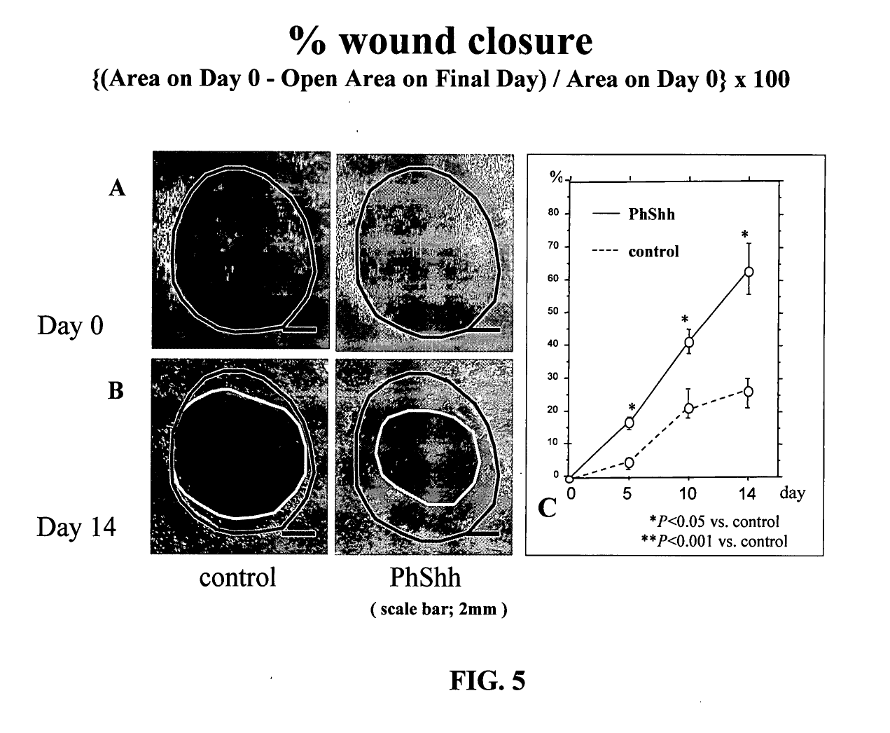

The results of a typical experiment are shown in FIG. 5. The appearance of wounds at day 0 in groups treated with phShh or control plasmid is shown in FIG. 5A. Referring to FIGS. 5A and B (left panels), a comparison of the open wound area at day 0 and day 14 in the control group showed a somewhat smaller open wound area, corresponding to about 25% wound closure. In marked contrast, the open area was much smaller than on day 0 in the phShh treated animals, corresponding to about 70% wound closure at this time. More particularly, the Shh treated wounds showed granulation of more than half of the wound area, compared with scant epithelialization in the controls. As a consequence, Shh treatment resulted in a significantly smaller wound within 5 days of treatment. A graphical comparison of the percentage of wound closure at 5, 10, and 14 days is shown in FIG. 5C. At each time point analyzed, the % wound closure was significantly accelerated in the phShh treated vs. control plasmid group (p.<0.05 at day 5; p.<0.005 at days 10 and 14). By day 14, this effect was highly significant (e.g., % wound closure; 25.0±2.0% vs. 65.3±7.6%, control vs. phShh, P<0.001, FIG. 5C).

### Example 12

### Topical Application of Hedgehog Gene to Skin Wounds Promotes Wound Healing as Assessed by Histological Criteria

Groups of mice were treated as described in Example 3 above. Ten days following wounding, the effects of topical phShh gene therapy were evaluated using macroscopic and microscopic criteria described above, including visual assessment of wound coloration, and microscopic analysis of degree of cellular invasion, formation of granulation tissue, vascularity of the wounded area and degree of re-epithelialization, in sections stained with hematoxylin and eosin (H&E) or Masson's trichrome (MT).

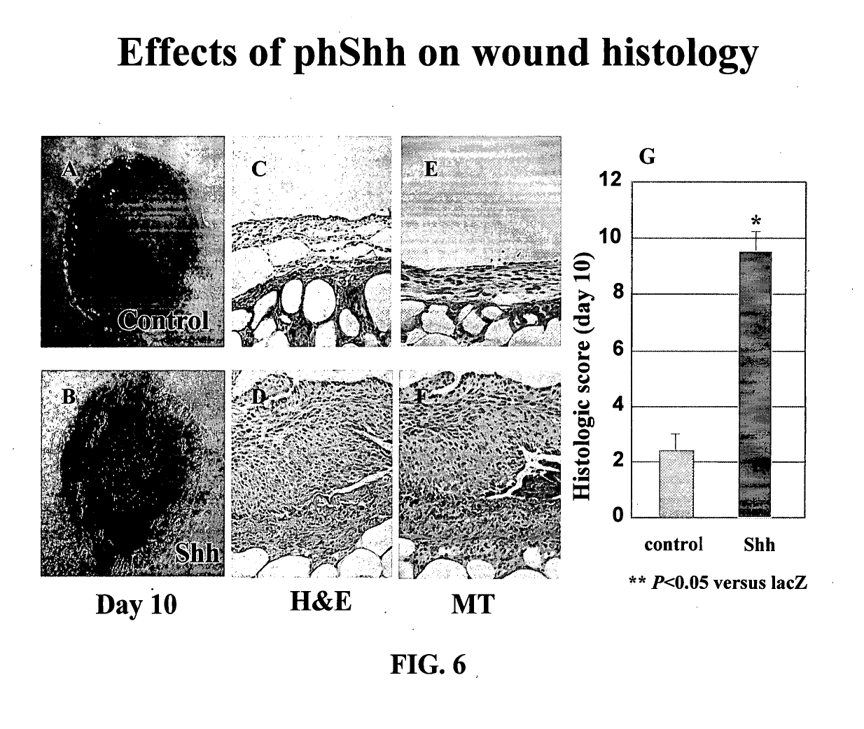

Macroscopic observations on day 10 are shown in FIGS. 6A and 6B. The gross appearance of the wounds in the phShh treated groups (FIG. 6B) was much more reddish than that of controls (FIG. 6A). The red appearance is thought to be due to the presence of granulation tissue, which is highly vascular. At the microscopic level, in sections stained with H&E, granulation tissues was observed to be much thicker than in the control groups (compare FIGS. 6C and 6D, and see FIG. 6G). A histological comparison of wounds treated with phShh and control plasmid is also shown following staining of sections with Masson's trichrome (FIGS. 6E, F). Consistent with the findings upon gross observation, histology revealed increased cellular infiltration, collagen deposition and thick granulation tissue in phShh-treated wounds. The histological score of wounds from mice treated with phShh was significantly higher (2.3±1.0 vs. 9.5±2.1, control vs. phShh, P<0.001, FIG. 6G).

### Example 13

### Topical Application of Hedgehog Gene to Skin Wounds Promotes Wound Healing by Increasing Vascularity

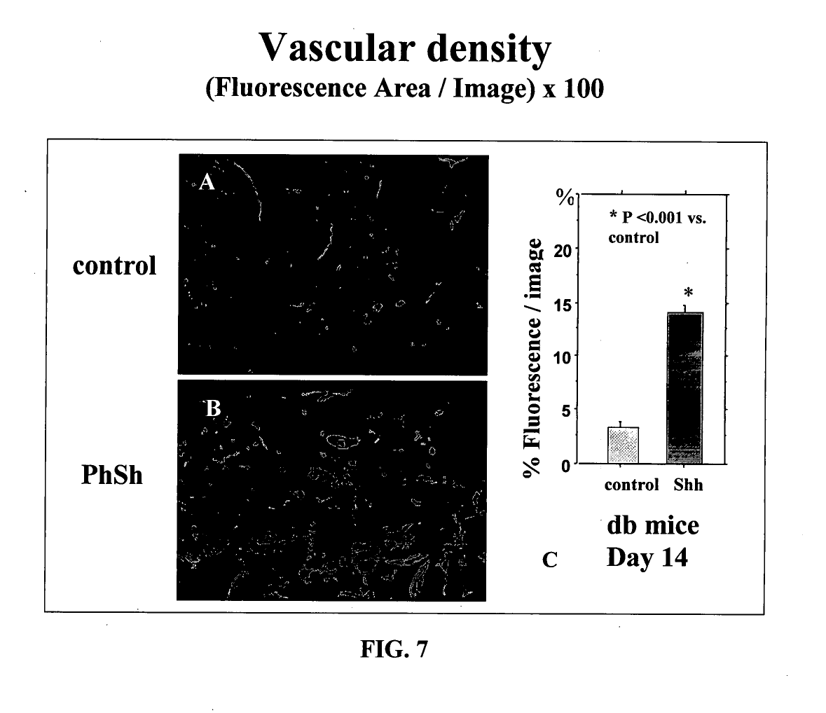

Groups of diabetic mice were subjected to wounding and topical application of vector containing Shh or control vector, and assayed as described above. On day 14 after treatment with either phShh or control plasmid, vascularity in the wounds was assessed by determining the percentage of fluorescent area observed per microscopic field, as described in Methods above. FIGS. 7A and 7B show the appearance of blood vessels in these tissues as visualized by fluorescence microscopy. Wound angiogenesis was analyzed in using fluorescent BS1 lectin in 10 μm frozen sections to visualize neovascularizarion in the resected wounds. From these images (results at 14 day shown), it is apparent that the density of vasculature (seen as fluorescent images) was much greater in wounds treated with phShh than with control plasmid. (FIGS. 7A, B). FIGS. 7A and 7B show a comparison of neovascularization at the wound margin in control (LacZ)s plasmid or phShh treated diabetic mice after 14 days. Compared to the control group, Shh treated wounds displayed significantly enhanced vascularity, with sprouting toward the center part of the wound. Shh significantly enhanced wound vascularity, as assessed by the percentage of the pixels in each image that were fluorescent (e.g., 4.79±0.32% vs. 14.63±0.901%, control vs. phShh, P<0.001).

### Example 14

### Topical Application of Hedgehog Gene to Skin Wounds Promotes Recruitment of Bone Marrow Stem/progenitor Cells into Wound Neovasculature

To examine whether hShh could promote migration of endothelial progenitor cells (EPCs) into skin wounds, wounds were prepared as described in Examples above using GFP/BMT mice. Plasmids (phShh or control) were added topically to the wounds as above. At 14 days after wounding, rhodamine conjugated BA1 lectin was injected and the skin wounds were harvested as described above. More particularly, to determine whether topical phShh augments recruitment of BM-derived EPCs into sites of wound repair, a chimeric mouse model (GFP/BMT) was selected. GFP/BMT mice are the recipients of bone marrow (BM) from donors in which the cells express GFP. Accordingly, the GFP label enables identification of cells derived from a BM progenitor cell. After recovery from the bone marrow transplantation (BMT) procedure, wounds were created on the dorsum of the mice and then randomly assigned to treatment with phShh or control plasmid. Fourteen days after wounding, the mice were sacrificed and wounds were harvested. To visualize functional the neovasculature in the healing wound, 5 animals of each group were injected rhodamine-conjugated BS1 lectin via the left ventricle before sacrifice.

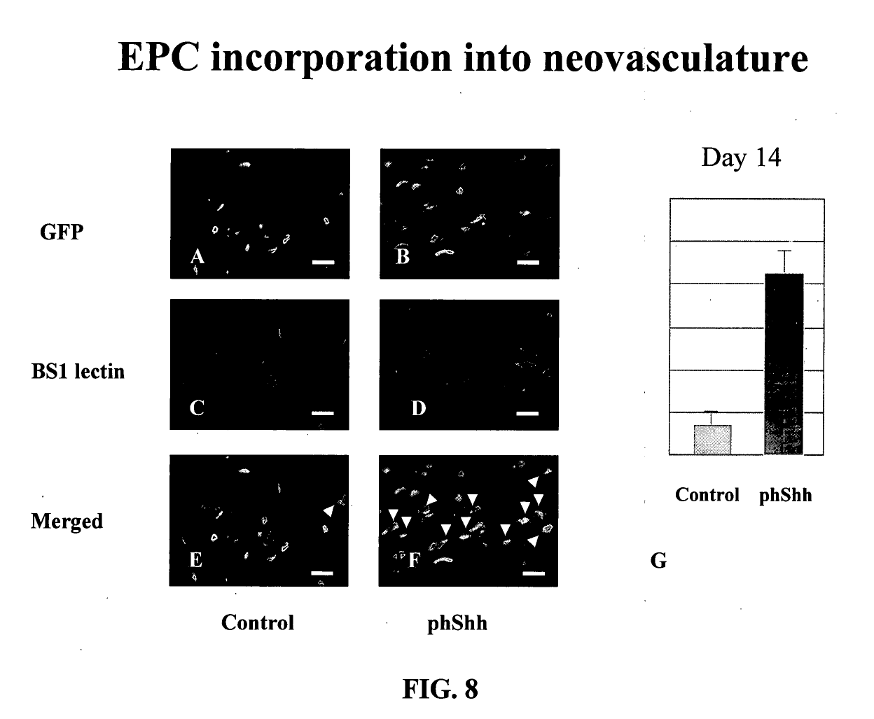

Results of this study are shown in FIG. 8. The contribution of BM-derived EPCs into the neovasculature of the healing wound was determined by fluorescence microscopy. Under observation using appropriate filter sets, red fluorescence identified BS1 lectin binding cells (endothelial cells) in functional vessels and green fluorescence identified GFP+ BM-derived cells. Double positive cells (merged images, white arrowheads) represented BM-derived EPCs. Cells labeled with GFP and BS1 lectin, respectively, are seen in FIGS. 8A, B and FIGS. 8C, D. The images in FIGS. 8E and 8F are merged to show cells tagged with both labels (i.e., GFP+ and lectin+).

In the group treated with control plasmid, few merged cells were seen. By contrast, many merged cells (indicated by arrowheads) were observed in the animals treated with phShh (compare FIGS. 8E and 8F). Many BM-derived EPCs were incorporated into the neovasculature, and notably, these double positive cells were found to be incorporated into microvasculature structures. Quantitation of the results is shown in FIG. 8G. It is seen that BM-derived EPC incorporation into the neovasculature in the phShh treated group was significantly increased compared to the control group (P<0.001, vs. control). For example, in a typical high power field, a mean of about 14 GFP+/BS1+ cells was present in wounds treated with control plasmid, whereas the mean numbers of these cells were about 43/field in the Shh treated group. The results of this study demonstrate that in response to topical application of phShh, stem/progenitor cells are recruited from the bone marrow and become incorporated into newly formed vasculature, including microvasculature in the granulation tissue of the wound.

### Example 15

### Shh Protein Upregulates mRNA Expression of Multiple Cytokines in Skin Cells

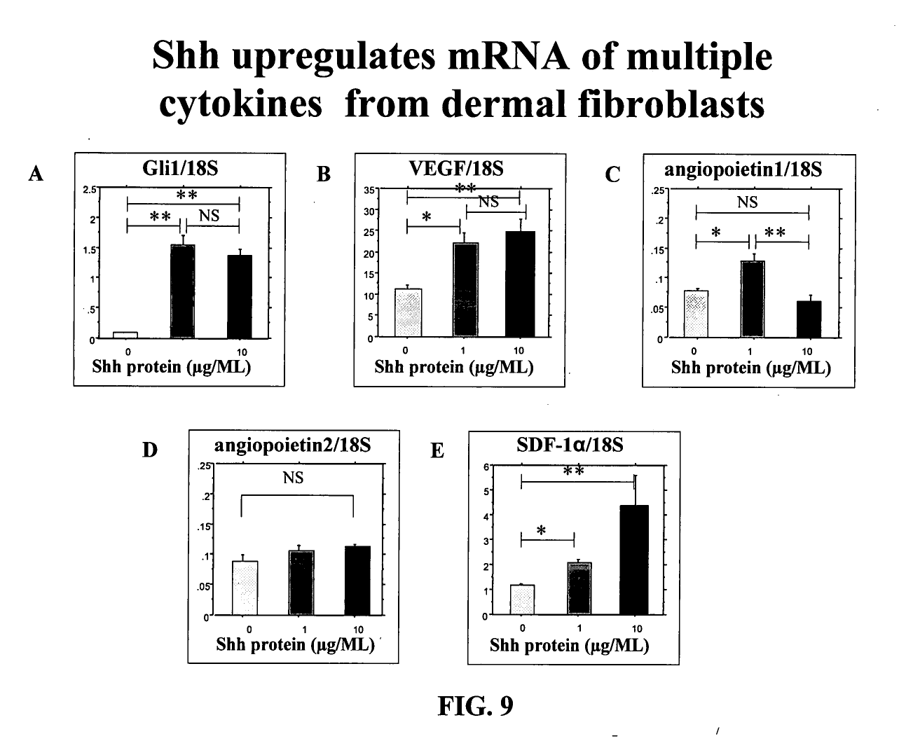

To identify potential mechanisms responsible for the therapeutic effect of Shh for wound healing, mRNA expression of a panel of candidate genes was evaluated in primary cultures of dermal fibroblasts, prepared as described above in Methods. Following treatment of the cells with Shh protein at 1 or 10 μg/mL, RT-PCR analysis was performed as described above to evaluate the presence of transcripts for the following cytokines: Gli-1, VEGF, angiopoietin 1, angiopoietin2, and SDF-1α. Referring to FIG. 9, it was found that treatment of the cells with Shh caused statistically significant upregulation of mRNA transcripts for Gli1 (FIG. 9A), VEGF (FIG. 9B), angiopoietin 1 (FIG. 9C), and SDF-1α (FIG. 9E).

Thus, the Example shows that Shh upregulates multiple cytokines from dermal fibroblasts. More specifically, there was upregulation of the Hh related transcription factor Gli1, indicating that the hedgehog pathway is intact in these adult cells. The expression of VEGF and angiopoietin-1 were upregulated by Shh, however the expression of angiopoietin-2 was not significantly altered. In addition, the expression of SDF-1, a trafficking chemokine for hematopoietic stem cells, was also increased.

It has been reported that Shh acts on mesenchymal cells and induces the secretion of multiple angiogenic cytokines including VEGF and angiopoietins. The Example confirms and extends these results to at least one cell type of the skin, i.e., fibroblasts.

### Example 16

### Shh Protein Promotes Proliferation of Skin Cells

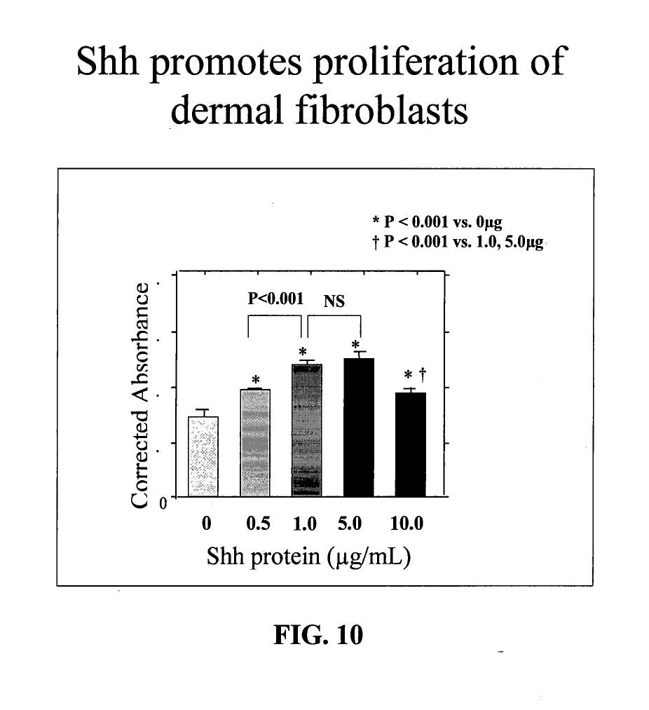

The effect of Shh on dermal fibroblast proliferation was examined. Primary cultures of dermal fibroblasts were prepared using standard techniques and treated with several concentrations of Shh protein (i.e., 0, 0.5, 1.0, 5.0, 10.0 μg/mL). Proliferation was determined by a cell proliferation assay (MTS assay). Results, shown in FIG. 10, revealed that Shh significantly promotes proliferation of dermal fibroblasts in a dose-dependent manner, up to 5.0 μg/mL (0 μg/ml 0.148±0.004, 0.5 μg/ml 0.187±0.007, 1 μg/ml 0.232±0.004, 5 μg/ml 0.243±0.007, 10 μg/ml 0.191±0.006, *P<0.0001 vs. 0 μg/ml).

### Example 17

### Shh Promotes Proliferation, Migration, Adhesion and Tube Formation by Endothelial Precursor Cells

Endothelial cells are known to express Ptc1, and there is recognition that hedgehog signaling is involved in endothelial tube formation [28,29]. This Example demonstrates that endothelial precursor cells (EPCs) also exhibit functional Ptc1 expression and respond to hedgehog stimulation by forming new blood vessels.

To detect Ptc1 expression of EPCs, cultured EPCs derived from NLS-Ptc1-lacZ mice were used for immunofluorescence analysis. EPCs were identified by co-staining with acLDL-DiI and FITC-conjugated isolectin B4, both of which are markers of endothelial lineage. Ptc1 expression of EPCs from NLS-Ptc1-lacZ mice was visualized as blue fluorescence using anti-galactosidase antibody.

Triple labeling for acLDL-DiI, FITC-conjugated isolectin B4 and .-gal was considered as evidence that EPCs express Ptc1.

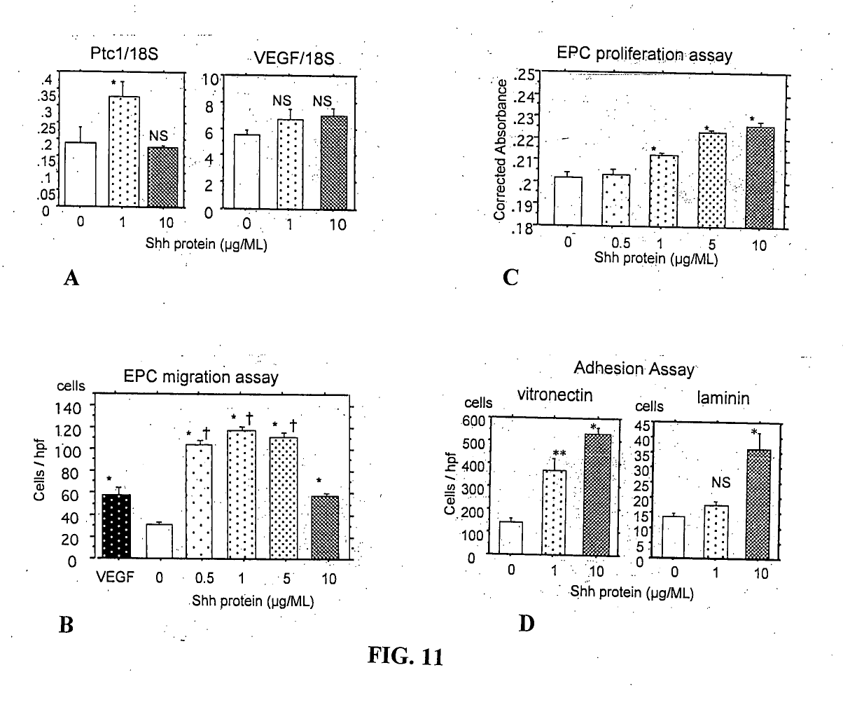

To examine the effects of Shh on EPCs, we first evaluated Ptc1 gene expression in these cells by RT-PCR. Referring to FIG. 11A, an increase of Ptc1 gene expression in EPCs was observed with 1 μg/ml of Shh supplementation and returned to baseline level with 10 μg/ml of Shh (0 μg/ml 0.19±0.046, 1 μg/ml 0.329±0.061, 10 μg/ml 0.178±0.006, *P<0.05 vs. 0 μg/ml). This result indicates that Gli-dependent Hh pathway may not be upregulated in a mono-phasic dose dependant manner. VEGF gene expression was not increased in EPCs by treatment with Shh (FIG. 11A).

We further evaluated the ability of Shh to induce EPC migration. More specifically, the migratory response of EPCs toward different dosages of Shh stimulation was measured using a modified Boyden chamber migration assay as described above. As shown in FIG. 11B, EPCs were induced to migrate in the presence of Shh. The effect of Shh on migration peaked at 1 μg/ml, whereas higher concentrations elicited less stimulation (*, P<0.001, vs. 0 μg/ml Shh). This effect was significantly greater than that of 50 ng/ml of VEGF (†t, P<0.001, vs. 50 ng/ml VEGF).

The effect of Shh on EPC proliferation was also examined by MTS assay (FIG. 11C). Shh increased proliferative activity in a dose dependant manner (0 μg/ml 0.202±0.002, 0.5 μg/ml 0.203±0.003, 1 μg/ml 0.212±0.001, 5 μg/ml 0.223±0.001, 10 μg/ml 0.226±0.002, *P<0.001 vs. 0 μg/ml).

To determine whether Shh alters EPC adhesion, adhesion assays were performed as described above. EPCs were incubated with Shh (0, 1, 10 μg/ml) for 36 h. and replated on vitronectin or laminin-coated dishes. Referring to FIG. 11C, the results of this study demonstrated that after replating, EPCs pre-exposed to Shh exhibited a significant increase in the number of adhesive cells at 30 minutes in a dose dependant manner (vitronectin; 0 μg/ml 141.8±18.012 cells, 1 μg/ml 371.4±49.878 cells, 10 μg/ml 532.8±24.897 cells, laminin; 0 μg/ml 13.6±1.166 cells, 1 μg/ml 17.4±1.435, 10 μg/ml 36.2±5.286, n=8, *, P<0.001, **, P<0.01 vs. 0 μg/ml).

Tube formation assays were performed to evaluate in vitro capillary morphogenesis induced by EPCs exposed to Shh. Observations by phase contrast microscopy of EPCs, for example in the presence or absence of 2 μg/ml Shh protein for 48 hours, revealed that EPCs formed tube structures following exposure to Shh. By contrast, tube formation was rare in the control treated EPCs.

Collectively the studies on the effects of Shh on EPC proliferation, migration, adhesion and tube formation demonstrate that this morphogen is a potent angiogenic factor that can promote formation of new microvasculature and hasten healing in wounded skin by stimulating bone marrow-derived EPCs to form new vessels.

### REFERENCES

It is believed that a review of the references will increase appreciation of the present invention. In some instances the references are prefaced by bracketed numbers which correspond to those in the preceding text.
Nusslein-Volhard C, W. E. Nature 287, 795-801 (1980).
Wang, M. et al. Nature Medicine 1, 1184-1188 (1995).
Roelink, H. et al. Cell 76, 761-755 (1994).
Goodrich, L. & Scott, M. Neuron 21, 1243-1257 (1998).
Johnson, R. L. & Tabin, C. J. Cell 90(6), 979-990 (1997).
Pepicelli, C. V., Lewis, P. M. & McMahon, A. P. Current Biology 8, 1083-1086 (1998).
Ramalho-Santos, M., Melton, D. A. & McMahon, A. P. Development 127, 2763-2772 (2000).
St-Jacques, B. et al. Curr Biol 8, 1058-68 (1998).
St-Jacques, B., Hammerschmidt, M. & McMahon, A. P. Genes Dev 13, 2072-86 (1999).
Pola, R. et al. Nature Medicine 7, 706-711 (2001).
Goodrich, L. V., Milenkovic, L., Higgins, K. M. & Scott, M. P. Science 277(5329), 1109-1113 (1997).
Zardoya, R., Abouheif, E. & Meyer, A. Trends Genet 12(12), 496-497 (1996).
Bitgood, M. J. & McMahon, Dev. Biol. 172(1), 126-138 (1995).
Kogerman, P. et al. Nature Cell Biology 1(5), 312-319 (1999).
Sisson, J. C., Ho, K. S., Suyama, K. & Scott, M. P. Cell 90(2), 235-245 (1997).
Pola, R. et al. Circulation 108, 479-85 (2003).
Roelink, H. et al. Cell 81(3), 445-455 (1995).
Porter, J. A. et al. Cell 86, 21-34 (1996).
Lee et al. Science 266:1528-1537(1994).
Porter et al. Nature 374:363-366 (1995).
Bumcrot D. A. et al. Mol. Cell Biol. 15:2294-2303 (1995).
Ekker S. C. et al. Curr. Biol. 5:944-955 (1995).
Lai C. J. et al. Development 121:2349-2360 (1995).
Ingham P W. EMBO J. 17:3505-3511 (1998).
[14] Goodson W H, 3rd, Hunt T K. Wound collagen accumulation in obese hyperglycemic mice. Diabetes. 1986; 35:491-5.
[15] Morshead C M, Benveniste P, Iscove N N, van der Kooy D. Hematopoicompetence is a rare property of neural stem cells that may depend on genetic and epigenetic alterations. Nat Med. 2002; 8:268-73.
[16] Teshima T, Ordemann R, Reddy P, Gagin S, Liu C, Cooke K R, Ferrara J L. Acute graft-versus-host disease does not require alloantigen expression on host epithelium. Nat Med. 2002; 8:575-81.
[17] Hansen S L, Myers C A, Charboneau A, Young D M, Boudreau N. HoxD3 accelerates wound healing in diabetic mice. Am J Pathol. 2003; 163:2421-31.
[18] Greenhalgh D C, Sprugel K M, Murray M J, Ross R. PDGF and FGF stimulate wound healing in the genetically diabetic mouse. Am J Pathol. 1990; 136:1235-46.
[19] Jacobi J, Jang J J, Sundram U, Dayoub H, Fajardo L F, Cooke J P. Nicotine accelerates angiogenesis and wound healing in genetically diabetic mice. Am J Pathol. 2002; 161:97-104.
[20] Asahara T, Murohara T, Sullivan A, Silver M, van der Zee R, Li T, Witzenbichler B, Schatteman G, Isner J M. Isolation of putative progenitor endothelial cells for angiogenesis. Science. 1997; 275:964-7.
[21] Asahara T, Takahashi T, Masuda H, Kalka C, Chen D, Iwaguro H, Inai Y, Silver M, Isner J M. VEGF contributes to postnatal neovascularization by mobilizing bone marrow-derived endothelial progenitor cells. EMBO J. 1999; 18:3964-3972.
[22] Shin D, Garcia-Cardena G, Hayashi S, Gerety S, Asahara T, Stavrakis G, Isner J, Folkman J, Gimbrone M A, Jr., Anderson D J. Expression of ephrinB2 identifies a stable genetic difference between arterial and venous vascular smooth muscle as well as endothelial cells, and marks subsets of microvessels at sites of adult neovascularization. Dev Biol. 2001; 230: 139-50.
[23] Mallat Z, Silvestre J S, Le Ricousse-Roussanne S, Lecomte-Raclet L, Corbaz A, Clergue M, Duriez M, Barateau V, Akira S, Tedgui A, Tobelem G, Chvatchko Y, Levy B I. Interleukin-18/interleukin-18 binding protein signaling modulates ischemia-induced neovascularization in mice hindlimb. Circ Res. 2002; 91:441-8.
[24] Taylor F R, Wen D, Garber E A, Carmillo A N, Baker D P, Arduini R M, Williams K P, Weinreb P H, Rayhorn P, Hronowski X, Whitty A, Day E S, Boriack-Sjodin A, Shapiro R I, Galdes A, Pepinsky R B. Enhanced potency of human Sonic hedgehog by hydrophobic modification. Biochemistry. 2001; 40:4359-71.
[25] Schratzberger P, Schratzberger G, Silver M, Curry C, Kearney M, Magner M, Alroy J, Adelman L S, Weinberg D H, Ropper A H, Isner J M. Favorable effect of VEGF gene transfer on ischemic peripheral neuropathy. Nat Med. 2000; 6:405-13.
[26] Walter D H, Rittig K, Bahlmann F H, Kirchmair R, Silver M, Murayama T, Nishimura H, Losordo D W, Asahara T, Isner J M. Statin therapy accelerates reendothelialization: a novel effect involving mobilization and incorporation of bone marrow-derived endothelial progenitor cells. Circulation. 2002; 105:3017-24.
[27] Goodrich L V, Milenkovic L, Higgins K M, Scott M P. Altered neural cell fate and medulloblastoma in mouse patched mutants. Science. 1997; 277(5329):1109-1113.
[28] Vokes S A, Yatskievych T A, Heimark R L, McMahon J, McMahon A P, Antin P B, Krieg P A. Hedgehog signaling is essential for endothelial tube formation during vasculogenesis. Development. 2004; 131:4371-80.
[29] Kanda S, Mochizuki Y, Suematsu T, Miyata Y, Nomata K, Kanetake H. Sonic hedgehog induces capillary morphogenesis by endothelial cells through phosphoinositide 3-kinase. J Biol. Chem. 2003; 278:8244-9.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for preventing, treating or reducing the severity of a wound or wounding disorder or accelerating wound healing in a mammal, the method comprising administering a therapeutically effective amount of at least one morphogenic protein or effective fragment thereof, or a nucleic acid encoding same.

2. The method of claim 1, wherein the method further comprises selecting a patient having a wound or wounding disorder and administering the nucleic acid or protein directly to or near a wound in need of treatment.

3. The method of claim 1, wherein the wound is an ulcer, a burn, a traumatic wound, or a surgical wound.

4. The method of claim 3, wherein the wound is an ulcer selected from the group consisting of a diabetic ulcer, an ulcer of vascular insufficiency and a pressure ulcer.

5. The method of claim 1, wherein the morphogenic protein is selected from the group consisting of human hedgehog (Shh) protein, human desert hedgehog (Dhh) protein and human Indian hedgehog (Ihh) protein.

6. The method of claim 5, wherein the nucleic acid encodes an N-terminal portion of the hedgehog protein.

7. The method of claim 1, further comprising administering a therapeutically effective amount of at least one of an angiogenic protein or a hematopoeitic protein, or a nucleic acid encoding same, and optionally at least one of endothelial cells (EC) or endothelial precursor cells (EPC).

8. A method for inducing new blood vessel formation in the skin of a mammal in need of such treatment comprising administering a therapeutically effective amount of at least one morphogenic protein or effective fragment thereof, or a nucleic acid encoding same.

9. The method of claim 8, wherein the skin of the mammal has been impacted by a wound or wounding disorder.

10. The method of claim 9, wherein the method further comprises expressing the morphogenic protein or fragment in or near a wound in the mammal to prevent or treat the wounding disorder.

11. A pharmaceutical product for preventing or treating a wound or wounding disorder or accelerating wound healing in a mammal, the product comprising at least one morphogenic protein or effective fragment thereof, or a nucleic acid encoding same, formulated to be physiologically acceptable to the mammal.

12. The pharmaceutical product of claim 11, further formulated for topical administration.

13. The pharmaceutical product of claim 12, formulated in a patch.

14. The pharmaceutical product of claim 12, formulated for sustained release.

15. The pharmaceutical product of claim 11, further comprising at least one of an angiogenic protein or a hematopoietic protein, or a nucleic acid encoding same, and optionally at least one of endothelial cells (EC) or endothelial precursor cells (EPC).

16. A kit for the administration of at least one morphogenic protein to the skin of a mammal, the kit comprising at least one morphogenic protein or effective fragment thereof, or a nucleic acid encoding same, the kit further comprising a pharmacologically acceptable carrier, and directions for using the kit.

17. The kit of claim 16, wherein the morphogenic protein or nucleic acid encoding same is formulated for topical administration.

18. The kit of claim 16, wherein the kit further comprises at least one of an angiogenic protein or a hematopoietic protein, or a nucleic acid encoding same, and optionally at least one of endothelial cells (EC) or endothelial precursor cells (EPC).

19. A method for increasing recruitment of endothelial precursor cells (EPCs) into blood vessels in the skin of a mammal, the method comprising contacting the skin of the mammal with an effective amount of at least one morphogenic protein or effective fragment thereof, or a nucleic acid encoding same.

20. A method for increasing production of at least one cytokine by skin cells of a mammal, comprising contacting the cells with an effective amount of at least one morphogenic protein or effective fragment thereof, or a nucleic acid encoding same.

21. The method of claim 20, wherein the cytokine is Glc-1, Ptc-1, vascular endothelial growth factor (VEGF), angiopoietin-1, or SDF-1α.

22. The method of claim 20, wherein the method is performed in vitro.

23. The method of claim 20, wherein the method is performed in vivo.

24. A method for increasing cell proliferation by skin cells of a mammal, comprising contacting the cells with an effective amount of at least one morphogenic protein or effective fragment thereof, or a nucleic acid encoding same.

25. The method of claim 24, wherein the method is performed in vitro.

26. The method of claim 24, wherein the method is performed in vivo.
